(19) United States
(12) Patent Application Publication
(10) Pub. No.: US 2007/0026067 A1
(43) Pub. Date: Feb. 1, 2007

(54) Liquid formulations for controlled delivery of benzisoxazole derivatives

(76) Inventors: Noymi V. Yam, Sunnyvale, CA (US); Nipun Davar, Fremont, CA (US); Gayatri Sathyan, San Jose, CA (US); Suneel K. Gupta, Sunnyvale, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/495,111

(22) Filed: Jul. 28, 2006

Related U.S. Application Data
(60) Provisional application No. 60/703,143, filed on Jul. 28, 2005.

(51) Int. Cl. A61K 9/22; A61K 31/519; A61K 31/42

(52) U.S. Cl. 424/468; 514/379; 514/259.41

(57) ABSTRACT

Disclosed are dosage forms including a controlled release dosing structure; and a liquid formulation contained within the controlled release dosing structure; wherein the liquid formulation comprises a benzisoxazole derivative and a liquid carrier. Also disclosed are methods of making and using such dosage forms.

### CROSS REFERENCE TO RELATED U.S. APPLICATIONS

The present application claims the benefit under 35 U.S.C. 119(e) of Provisional application 60/703,143 filed Jul. 28, 2005.

### FIELD OF THE INVENTION

The invention relates to dosage forms and methods comprising benzisoxazole derivatives. More particularly, the invention relates to dosage forms, methods, and new uses of benzisoxazole derivatives having enhanced bioavailability.

### BACKGROUND

Patients presenting with psychosis can show a reduction in their symptoms after treatment with antipsychotic drugs. Traditional antipsychotic drugs were effective with some patients, but exhibited a wide range of undesirable side effects. Such side effects include parkinsonism, akathisia, acute dystonia, and tardive dyskinesia.

A class of newer antipsychotic drugs, referred to as atypical antipsychotics, have been introduced more recently. One of the benefits of atypical antipsychotics is a reduced side effect profile. However, even with the reduction in the side effect profile, undesirable side effects remain, including but not limited to orthostatic hypotension, seizures, dysphagia, and hyperprolactinemia. Examples of atypical antipsychotics include risperidone, olanzapine, and clozapine.

Risperidone is an antipsychotic agent indicated for the management of manifestations of psychotic disorders. Risperidone belongs to the chemical class of benzisoxazole derivatives. Physicians' Desk Reference, Thompson Healthcare, 56th Ed., pp. 1796-1800 (2002). Risperidone is a potent antagonist of the serotonin 5-HT2 receptor and the dopamine D2 receptor. Risperidone is also a selective antagonist at the alpha1 and alpha2 adrenergic receptors.

An immediate release tablet containing risperidone is currently marketed as Risperdal® by Janssen Pharmaceutical Products, L. P. Physicians' Desk Reference, Thompson Healthcare, 56th Ed., pp. 1796-1800 (2002). A long-lasting injectible for risperidone, Risperdal® Consta™, is also being marketed.

Paliperidone is the major active metabolite of risperidone. Risperidone is extensively metabolized in the liver to an equipotent metabolite, paliperidone, and the sum of the two compounds (active moiety) is thought to provide the clinical effect of risperidone. Paliperidone shares the characteristic D2, 5HT2A antagonism of atypical antipsychotic drugs, and a receptor-bind profile similar to risperidone. Humans can be phenotyped as (a) poor, (b) intermediate or (c) extensive risperidone metabolizers on the basis of their metabolic ratio (e.g., the ratio of urine recovery of risperidone to that of paliperidone over a period of 8 hours after oral intake of 10 mg of risperidone). The pharmacological profile of paliperidone closely resembles that of risperidone itself. Paliperidone is more fully described in U.S. Pat. No. 5,158,952. Additional compounds are disclosed in U.S. Pat. Nos. 4,804,665 and 4,458,076.

Risperidone and paliperidone are practically insoluble in water. Additionally, since paliperidone has a long half-life of about one day, it is not a typical candidate for extended delivery. Risperidone has a shorter half-life but since it metabolizes to paliperidone, one can say the active moiety has a longer half-life. Side effects associated with administration of paliperidone are similar to those associated with administration of risperidone.

The low solubility of benzisoxazole derivatives such as risperidone and paliperidone creates problems for formulating these compounds into dosage forms, including dosage forms comprising controlled delivery dosing structure. Accordingly, there remains a need for effective dosing methods, dosage forms and devices that will permit the dosing of benzisoxazole derivatives in a way that overcomes the low solubility of such derivatives. Exemplary methodologies, dosage forms, methods of preparing such dosage forms and methods of using such dosage forms are disclosed herein.

### SUMMARY OF THE INVENTION

In an aspect, the invention relates to dosage forms comprising: a controlled release dosing structure; and a liquid formulation contained within the controlled release dosing structure; wherein the liquid formulation comprises a benzisoxazole derivative and a liquid carrier.

In another aspect, the invention relates to methods comprising: providing a dosage form that comprises a controlled release dosing structure; providing a liquid formulation within the controlled release dosing structure, wherein the liquid formulation comprises a benzisoxazole derivative and a liquid carrier; and causing the controlled release dosing structure to controllably release the liquid formulation.

### BRIEF DESCRIPTION OF THE DRAWINGS

FIG. 1 shows in vitro performance of risperidone formulations

FIG. 2 shoes in vitro performance of paliperidone formulations

FIG. 3 shows results of release rate testing of dosage forms according to the invention.

FIG. 4 shows results of release rate testing of dosage forms according to the invention.

FIG. 5 shows a hard capsule dosage form according to the invention.

FIG. 6 shows a soft capsule dosage form according to the invention.

### DETAILED DESCRIPTION OF THE INVENTION

Enhanced Bioavailability using Liquid Formulations Versus Solid Formulations

The inventors have unexpectedly discovered that dosage forms and methods that use certain liquid formulations can provide enhanced bioavailability of benzisoxazole derivatives. Without wishing to be bound by a specific mechanism, the inventors have reasoned that solubilization or dispersion of the benzisoxazole derivatives in liquid formulations, and particularly in formulations containing surface-active carriers, may enhance solubility of the drug in-situ and therefore may provide the means to increase oral bioavailability.

The results of this discovery can be seen in the results disclosed in the Examples herein, and as further discussed below.

The present invention thus accomplishes an object of the invention of providing effective dosing methods, dosage forms and devices that will permit the dosing of benzisoxazole derivatives in a way that provides enhanced bioavailability. Of particular importance is the discovery that there are selected dosing structures or controlled releasing means, and equivalents thereof, that accomplish an object of the invention.

The bioavailability of risperidone and paliperidone in both dosage forms that comprise a liquid formulation and solid controlled release (CR) dosage forms have been studied relative to their respective immediate release dosage forms and is reported below in Examples 12-14. The observed relative bioavailability values as compared to an immediate release solution for both the solid and liquid CR formulation is summarized in Table 1. The bioavailability of drug was lower for all of the dosage forms listed in Table 1 as compared to the immediate release solution.

For non-disintegrating systems such as OROS™ it is believed that the system reaches the colon in about 3-5 hours. A. J. Coupe et al., Nocturnal scintigraphy imaging to investigate the gastrointestinal transit of dosage forms. Journal of Controlled Release 20:155-162 (1992); S. S. Davis et al., Transit of pharmaceutical dosage forms through the small intestine. Gut 27:886-892 (1986). For risperidone, a colonic intubation study revealed a lower bioavailability from the colon (52.5-60%) when a drug solution was introduced into the colon as compared to upper gastrointestinal tract administration (see Example 11). When a CR formulation is administered the drug is released throughout the gastrointestinal tract. Hence the availability of the drug release in the upper GI would be expected to be 100% relative to immediate release formulation and the availability of the drug would be lower for the amount of drug released in the large intestine (colon).

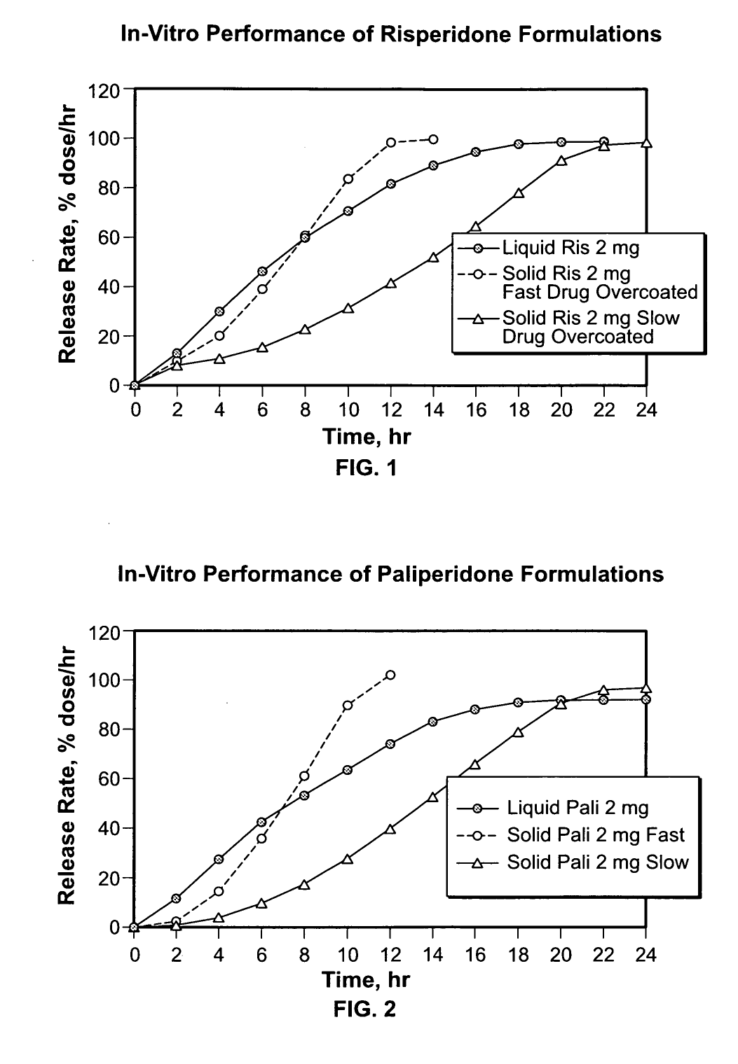

FIG. 1 shows the cumulative percent of drug released from the risperidone CR formulations of Examples 1, 9, and 10. Release rates were determined generally according to the method of Example 5. The cumulative percent drug released is similar for the dosage form comprising liquid formulation (38%) and the solid (fast) controlled release formulations (29.5%). Further drug release from the dosage form comprising liquid formulation is slower and is released for a longer time, which means more amount of drug is likely reaching the distal colon as compared to the solid (fast) CR dosage form. However, the overall relative bioavailability was higher with the dosage form comprising liquid formulation. This suggests that the colonic bioavailbility is likely to be higher with the dosage form comprising liquid formulation.

The colonic availability relative to immediate release formulation was estimated from the CR dosage forms as follows:

For dosage form comprising liquid formulation:

1. Assumption that drug released in 0-5 hours is 100% available=38%

2. Remaining portion of Overall relative BA i.e 65.6%−38.0%=27.6%

3. Percent drug released in 5-24 h=62%

Colonic availability therefore is estimated to be 27.6*100/62%=44.5%

Table 1 summarizes the estimated colonic bioavailabity of risperidone from risperidone CR formulations relative to the IR formulation. The availability from the dosage form comprising liquid formulation was estimated to be higher than that of the two solid comparison CR dosage forms.

Similar calculations were done for dosage forms comprising liquid formulation of paliperidone and two solid CR dosage forms of paliperidone. Release rates were determined generally according to the method of Example 5. The outcome is summarized in Table 2, and FIG. 2, and is consistent with the observations for the risperidone formulations. The bioavailability of drug from the dosage forms comprising liquid formulation of paliperidone appears to be higher than the bioavailability of drug from the solid CR dosage forms.

TABLE 1
Estimate of Colonic Bioavailability of Risperidone from
Risperidone Dosage Forms Relative to IR Solution

| | | | | | Estimate |
|---|---|---|---|---|---|
| | | | | | of Colonic |
| | | | | | Availability |
| | | Overall | In vitro Release | | from CR |
| Example | Type of | Relative | (%)^{b} | | Formulations |
| # | Formulation | BA^{a} | 0-5 h | 5-24 h | (%) |
| 1 | Liquid | 65.6 | 38.0 | 62.0 | 44.5 |
| 9 | Solid- | 54.5 | 29.5 | 70.5 | 35.0 |
| | Fast CR | | | | |
| 10 | Solid- | 41.8 | 13.0 | 87.0 | 33.0 |
| | Slow CR | | | | |

^{a}Relative BA = Bioavailability relative to immediate release solution
^{b}FIG. 1 - interpolated between 4 and 6 h time point

TABLE 2
Estimate of Colonic Bioavailability of Paliperidone from
Paliperidone Dosage Forms Relative to IR Solution

| | | | | | Estimate |
|---|---|---|---|---|---|
| | | | | | of Colonic |
| | | | | | Availability |
| | | Overall | In vitro Release | | from CR |
| Example | Type of | Relative | (%)^{b} | | Formulations |
| # | Formulation | BA^{a} | 0-5 h | 5-24 h | (%) |
| 2 | Liquid | 62.5 | 35.1 | 64.9 | 40.0 |
| 8 | Solid- | 52.0 | 25.4 | 74.6 | 36.0 |
| | Fast CR | | | | |
| 7 | Solid- | 34.0 | 7.1 | 92.9 | 29.0 |
| | Slow CR | | | | |

^{a}Relative BA = Bioavailability relative to immediate release solution
^{b}FIG. 2 - interpolated between 4 and 6 h time point

The invention will now be described in more detail below.

Definitions

All percentages are weight percent unless otherwise noted.

All publications cited to herein are incorporated by reference in their entirety and for all purposes as if reproduced fully herein.

The present invention is best understood by reference to the following definitions, the drawings and exemplary disclosure provided herein.

“Administering” or “administration” means providing a drug to a patient in a manner that is pharmacologically useful.

“Antioxidants” means a material that prevents or reduces the oxidation of other materials. Various kinds of antioxidants useful in the practice of the invention are discussed further elsewhere herein.

“Area under the curve” or “AUC” is the area as measured under a plasma drug concentration curve. Often, the AUC is specified in terms of the time interval across which the plasma drug concentration curve is being integrated, for instance AUC_{start-finish}. Thus, AUC₀₋₄₈ refers to the AUC obtained from integrating the plasma concentration curve over a period of zero to 48 hours, where zero is conventionally the time of administration of the drug or dosage form comprising the drug to a patient. AUCₜ refers to area under the plasma concentration curve from hour 0 to the last detectable concentration at time t, calculated by the trapezoidal rule. AUC_{inf} refers to the AUC value extrapolated to infinity, calculated as the sum of AUCₜ and the area extrapolated to infinity, calculated by the concentration at time t (Ct) divided by k. (If the t_{1/2} value was not estimable for a subject, the mean t_{1/2} value of that treatment was used to calculate AUC_{inf}.).

“Benzisoxazole derivative” or “drug” means risperidone and/or pharmaceutically acceptable salt(s) thereof, and/or paliperidone and/or pharmaceutically acceptable salt(s) thereof, and combinations of any of the above. In a preferred embodiment, the benzisoxazole derivative is present in the dosage form in an amount ranging from about 0.1 mg to about 20 mg; more preferably the benzisoxazole derivative is present in an amount ranging from about 0.1 mg to about 5 mg.

“Controlled release” and/or “controllably releasing” mean to release a dose of a benzisoxazole derivative into a surrounding environment at a predetermined rate of release for a prolonged period.

“Controlled release dosing structure” means a structure that, when in operation, serves to controllably release a dose of a benzisoxazole derivative into a surrounding environment.

“Dosage form” means a benzisoxazole derivative in a medium, carrier, vehicle, or device suitable for administration to a patient. “Oral dosage form” means a dosage form suitable for oral administration.

“Liquid formulation” means that mixture (i) that includes one or more benzisoxazole derivatives, one or more liquid carriers, and optionally other substances, and (ii) that is contained within the controlled released dosing structure and is controllably released when the dosage form operates to deliver the liquid formulation.

“Liquid carrier” means lipophilic solvents (e.g., oils and lipids), surfactants, and hydrophilic solvents, and/or mixtures thereof, that are useful for dissolving or suspending benzisoxazole derivatives in a form suitable for delivery to a patient. Various kinds of liquid carriers useful in the practice of the invention are discussed further elsewhere herein.

“Immediate-release dosage form” means a dosage form that releases greater than or equal to about 80% of the drug in less than or equal to about 1 hour following administration of the dosage form to a patient.

“Osmotic dosage form” means a dosage form that operates via an osmotic mechanism to release liquid formulations that benzisoxazole derivative(s) into a surrounding environment.

“Patient” means an animal, preferably a mammal, more preferably a human, in need of therapeutic intervention.

“Pharmaceutically acceptable salt” means any salt whose anion does not contribute significantly to the toxicity or pharmacological activity of the salt, and, as such, they are the pharmacological equivalents of the base of the benzisoxazole derivative. Suitable pharmaceutically acceptable salts include acid addition salts which may, for example, be formed by reacting the drug compound with a suitable pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid.

Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

“Pharmacologically active metabolites” means pharmacologically active metabolites of benzisoxazole derivatives.

“Prolonged period of time” means a continuous period of time of greater than about 2 hours, preferably, greater than about 4 hours, more preferably, greater than about 8 hours, more preferably greater than about 10 hours, more preferably still, greater than about 14 hours, most preferably, greater than about 14 hours and up to about 24 hours.

“Rate of release” or “release rate” means the quantity of benzisoxazole derivative released from a dosage form per unit time, e.g., milligrams of drug released per hour (mg/hr). Drug release rates for dosage forms may be measured as an in vitro rate of drug release, i.e., a quantity of drug released from the dosage form per unit time measured under appropriate conditions and in a suitable fluid.

The release rates referred to herein are determined by placing a dosage form to be tested in de-ionized water in metal coil or metal cage sample holders attached to a USP Type VII bath indexer in a constant temperature water bath at 37° C. Aliquots of the release rate solutions, collected at pre-set intervals, are then injected into a chromatographic system fitted with an ultraviolet or refractive index detector to quantify the amounts of drug released during the testing intervals.

As used herein a drug release rate obtained at a specified time refers to the in vitro release rate obtained at the specified time following implementation of the release rate test. The time at which a specified percentage of the drug within a dosage form has been released from said dosage form may be referred to as the “Tx” value, where “x” is the percent of drug that has been released. For example, a commonly used reference measurement for evaluating drug release from dosage forms is the time at which 70% of drug within the dosage form has been released. This measurement is referred to as the “T70” for the dosage form.

“Relative bioavailability” means

$$\frac{{AUC}_{inf} for inventive dosage form}{{AUC}_{inf} for immediate release dosage form}$$

wherein both dosage forms comprise the same or substantially the same amount of drug, expressed in units of mass.
   Dosage Forms

Various types of dosage forms are useful in the practice of this invention; it will be appreciated that the dosage forms described herein are merely exemplary. Generally, any dosage form that is capable of delivering liquid formulations is useful in the practice of this invention. Examples of dosage forms useful in the practice of this invention comprise liquid gelcaps, ORADUR® capsules (available from DURECT Corporation), and osmotic liquid dosage forms. In a preferable embodiment, the dosage form is an oral dosage form. In another preferable embodiment, the dosage form is a suppository, more preferably a vaginal suppository, or a rectal suppository. In yet another preferable embodiment, the dosage form is an implantable dosage form, such as a subcutaneous implant dosage form. An example of such a dosage form comprises DUROS® dosage forms, manufactured by ALZA Corp. (Mountain View Calif.).

In an embodiment, the dosage forms comprise osmotic dosage forms. Osmotic dosage forms for delivering liquid formulations and methods of using them are known in the art, for example, as described and claimed in the following U.S. Pat. Nos. 6,419,952; 6,174,547; 6,551,613; 5,324,280; 4,111,201; and 6,174,547. Methods of using oral osmotic devices for delivering therapeutic agents at an ascending rate of release can be found in International Application Numbers WO 98/06380, WO 98/23263, and WO 99/62496.

The present invention provides liquid formulation(s) for use with the inventive dosage forms. Generally, the inventive liquid formulations comprise liquid carriers. Exemplary liquid carriers for the present invention include lipophilic solvents (e.g., oils and lipids), surfactants, and hydrophilic solvents. Exemplary lipophilic solvents, for example, include, but are not limited to, Capmul PG-8, Caprol MPGO, Capryol 90, Plurol Oleique CC 497, Capmul MCM, Labrafac PG, N-Decyl Alcohol, Caprol 10G10O, Oleic Acid, Vitamin E, Maisine 35-1, Gelucire 33/01, Gelucire 44/14, Lauryl Alcohol, Captex 355EP, Captex 500, Capylic/Caplic Triglyceride, Peceol, Caprol ET, Labrafil M2125 CS, Labrafac CC, Labrafil M 1944 CS, Captex 8277, Myvacet 9-45, Isopropyl Nyristate, Caprol PGE 860, Olive Oil, Plurol Oleique, Peanut Oil, Captex 300 Low C6, and Capric Acid.

Exemplary surfactants for example, include, but are not limited to, Vitamin E TPGS, Cremophor (grades EL, EL-P, and RH40), Labrasol, Polysorbate (grades 20, 60, 80), Pluronic (grades L-31, L-35, L-42, L-64, and L-121), Acconon S-35, Solutol HS-15, and Span (grades 20, and 80). Exemplary hydrophilic solvents for example, include, but are not limited to, Isosorbide Dimethyl Ether, Polyethylene Glycol (PEG grades 300, 400, 600, 3000, 4000, 6000, and 8000) and Propylene Glycol (PG).

The skilled practitioner will understand that any formulation comprising a sufficient dosage of benzisoxazole derivative solubilized in a liquid carrier suitable for administration to a subject and for use in an osmotic device can be used in the present invention. In one exemplary embodiment of the present invention, the liquid carrier is PG, Solutol, Cremophor EL, or a combination thereof.

The liquid formulation according to the present invention can also comprise, for example, additional excipients such as an antioxidant, permeation enhancer and the like. Antioxidants can be provided to slow or effectively stop the rate of any autoxidizable material present in the capsule. Representative antioxidants can comprise a member selected from the group of ascorbic acid; alpha tocopherol; ascorbyl palmitate; ascorbates; isoascorbates; butylated hydroxyanisole; butylated hydroxytoluene; nordihydroguiaretic acid; esters of garlic acid comprising at least 3 carbon atoms comprising a member selected from the group consisting of propyl gallate, octyl gallate, decyl gallate, decyl gallate; 6-ethoxy-2,2,4-trimethyl-1,2-dihydro-guinoline; N-acetyl-2,6-di-t-butyl-p-aminophenol; butyl tyrosine; 3-tertiarybutyl 4-hydroxyanisole; 2-tertiary-butyl-4-hydroxyanisole; 4-chloro-2,6-ditertiary butyl phenol; 2,6-ditertiary butyl p-methoxy phenol; 2,6-ditertiary butyl-p-cresol: polymeric antioxidants; trihydroxybutyro-phenone physiologically acceptable salts of ascorbic acid, erythorbic acid, and ascorbyl acetate; calcium ascorbate; sodium ascorbate; sodium bisulfite; and the like. The amount of antioxidant used for the present purposes, for example, can be about 0.001% to 25% of the total weight of the composition present in the lumen. Antioxidants are known to the prior art in U.S. Pat. Nos. 2,707,154; 3,573,936; 3,637,772; 4,038,434; 4,186,465 and 4,559,237, each of which is hereby incorporated by reference in its entirety for all purposes.

The inventive liquid formulation can comprise permeation enhancers that facilitate absorption of the drug in the environment of use. Such enhancers can, for example, open the so-called “tight junctions” in the gastrointestinal tract or modify the effect of cellular components, such a p-glycoprotein and the like. Suitable enhancers can include alkali metal salts of salicyclic acid, such as sodium salicylate, caprylic or capric acid, such as sodium caprylate or sodium caprate, and the like. Enhancers can include, for example, the bile salts, such as sodium deoxycholate. Various p-glycoprotein modulators are described in U.S. Pat. Nos. 5,112,817 and 5,643,909. Various other absorption enhancing compounds and materials are described in U.S. Pat. No. 5,824,638. Enhancers can be used either alone or as mixtures in combination with other enhancers.

In certain embodiments, the inventive substances are administered as a self-emulsifying formulation. Like the other liquid carriers, the surfactant functions to prevent aggregation, reduce interfacial tension between constituents, enhance the free-flow of constituents, and lessen the incidence of constituent retention in the dosage form. The emulsion formulation of this invention comprises a surfactant that imparts emulsification. Exemplary surfactants can also include, for example, in addition to the surfactants listed above, a member selected from the group consisting of polyoxyethylenated castor oil comprising ethylene oxide in the concentration of 9 to 15 moles, polyoxyethylenated sorbitan monopalmitate, mono and tristearate comprising 20 moles of ethylene oxide, polyoxyethylenated sorbitan monostearate comprising 4 moles of ethylene oxide, polyoxyethylenated sorbitan trioleate comprising 20 moles of ethylene oxide, polyoxyethylene lauryl ether, polyoxyethylenated stearic acid comprising 40 to 50 moles of ethylene oxide, polyoxyethylenated stearyl alcohol comprising 2 moles of ethylene oxide, and polyoxyethylenated oleyl alcohol comprising 2 moles of ethylene oxide. The surfactants may be available from Atlas Chemical Industries.

In an embodiment, the liquid formulations of the present invention can initially comprise an oil and a non-ionic surfactant. The oil phase of the emulsion comprises any pharmaceutically acceptable oil that is not immiscible with water. The oil can be an edible liquid such as a non-polar ester of an unsaturated fatty acid, derivatives of such esters, or mixtures of such esters. The oil can be vegetable, mineral, animal or marine in origin. Examples of non-toxic oils can also include, for example, in addition to the surfactants listed above, a member selected from the group consisting of peanut oil, cottonseed oil, sesame oil, corn oil, almond oil, mineral oil, castor oil, coconut oil, palm oil, cocoa butter, safflower, a mixture of mono- and diglycerides of 16 to 18 carbon atoms, unsaturated fatty acids, fractionated triglycerides derived from coconut oil, fractionated liquid triglycerides derived from short chain 10 to 15 carbon atoms fatty acids, acetylated monoglycerides, acetylated diglycerides, acetylated triglycerides, olein known also as glyceral trioleate, palmitin known as glyceryl tripalmitate, stearin known also as glyceryl tristearate, lauric acid hexylester, oleic acid oleylester, glycolyzed ethoxylated glycerides of natural oils, branched fatty acids with 13 molecules of ethyleneoxide, and oleic acid decylester. The concentration of oil, or oil derivative in the liquid formulation can be from about 1 wt % to about 40 wt %, with the wt % of all constituents in the emulsion preparation equal to 100 wt %. The oils are disclosed in Pharmaceutical Sciences by Remington, 17th Ed., pp. 403-405, (1985) published by Mark Publishing Co., in Encyclopedia of Chemistry, by Van Nostrand Reinhold, 4th Ed., pp. 644-645, (1984) published by Van Nostrand Reinhold Co.; and in U.S. Pat. No. 4,259,323.

The amount of benzisoxazole derivative incorporated in the dosage forms of the present invention is generally from about 10% to about 90% by weight of the composition depending upon the therapeutic indication and the desired administration period, e.g., every 12 hours, every 24 hours, and the like. Depending on the dose of benzisoxazole derivative desired to be administered, one or more of the dosage forms can be administered.

The dosage form according to the present invention can also comprise, for example, controlled release structures such as an enteric coating used with or without the osmotic element of controlled delivery. The enteric coating can be applied onto the dosage form with or without other semipermeable membrane to achieve an effective delay in onset of drug release. Representative excipients for formation of the enteric coating include cellulose acetate phthalate, hydroxypropylmethylcellulose phthalate, copolymers of methacrylic acid and acrylic acid esters, and the like. Enteric coating formulations may contain plasticizers. The plasticizer may include triethylcitrate, glyceryltriacetate, acetyltriethylcitrate, dibutyl sebacate, diethylphthalate, polyethylene glycol having a molecular weight in the range of 200 to 8000, glycerol, castor oil, copolymers of propylene oxide and ethylene oxide, or mixtures thereof. Preferably, the plasticizer comprises 0% to about 20% by weight of the coating composition. Enteric coating formulations may also contain secondary film formers to increase mechanical robustness of the coating. The secondary film former may include xanthan gum, sodium alginate, propylene glycol alginate, hydroxypropylmethylcellulose (HPMC), hydroxyethylecellulose (HEC), sodium carboxymethylcellulose (sodium CMC), polyvinylpyrrolidone (PVP), carrageenan, other film-forming polymer or mixtures thereof. Preferably, the amount of secondary film former in the coating composition ranges from 0% to about 5% by weight of the dry coating composition. The application of the enteric coating can be achieved by using conventional coating processes, both aqueous and solvent-based. Procedures for the application of the enteric coatings are disclosed, among other places in U.S. Pat. No. 4,287,221, U.S. Pat. No. 6,420,473.

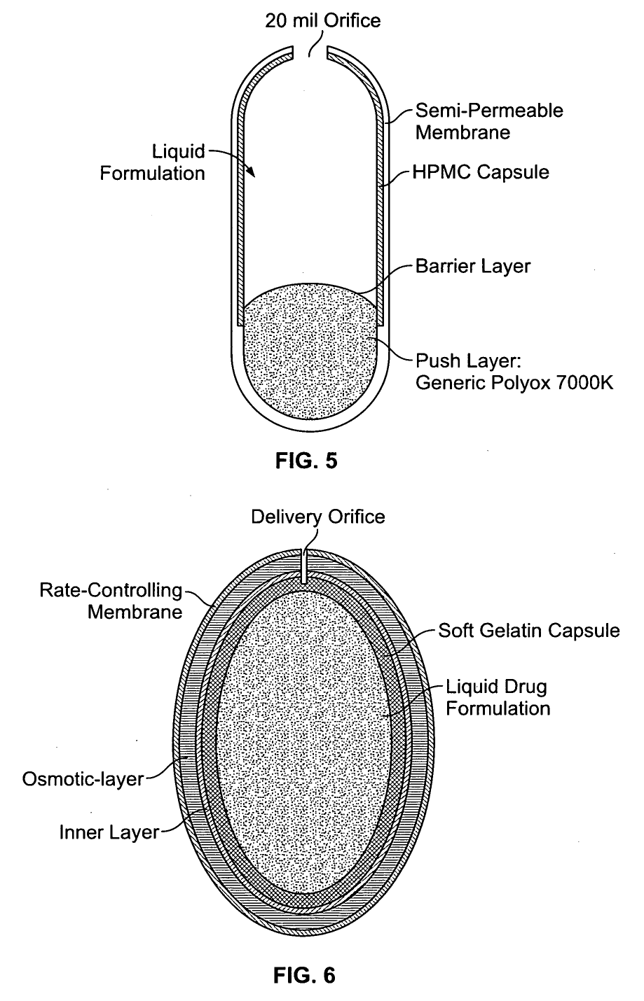

The osmotic dosage forms of the present invention can possess two distinct forms, a soft capsule form (shown in FIG. 6) and a hard capsule form (shown in FIG. 5). The soft capsule, as used by the present invention, preferably in its final form comprises one piece. The one-piece capsule is of a sealed construction encapsulating the drug formulation therein. The capsule can be made by various processes including the plate process, the rotary die process, the reciprocating die process, and the continuous process. An example of the plate process is as follows. The plate process uses a set of molds. A warm sheet of a prepared capsule lamina-forming material is laid over the lower mold and the formulation poured on it. A second sheet of the lamina-forming material is placed over the formulation followed by the top mold. The mold set is placed under a press and a pressure applied, with or without heat, to form a unit capsule. The capsules are washed with a solvent for removing excess agent formulation from the exterior of the capsule, and the air-dried capsule is encapsulated with a semipermeable wall. The rotary die process uses two continuous films of capsule lamina-forming material that are brought into convergence between a pair of revolving dies and an injector wedge. The process fills and seals the capsule in dual and coincident operations. In this process, the sheets of capsule lamina-forming material are fed over guide rolls, and then down between the wedge injector and the die rolls. The agent formulation to be encapsulated flows by gravity into a positive displacement pump. The pump meters the agent formulation through the wedge injector and into the sheets between the die rolls. The bottom of the wedge contains small orifices lined up with the die pockets of the die rolls. The capsule is about half-sealed when the pressure of pumped agent formulation forces the sheets into the die pockets, wherein the capsules are simultaneously filled, shaped, hermetically sealed and cut from the sheets of lamina-forming materials. The sealing of the capsule is achieved by mechanical pressure on the die rolls and by heating of the sheets of lamina-forming materials by the wedge. After manufacture, the agent formulation-filled capsules are dried in the presence of forced air, and a semipermeable lamina encapsulated thereto.

The reciprocating die process produces capsules by leading two films of capsule lamina-forming material between a set of vertical dies. The dies as they close, open, and close perform as a continuous vertical plate forming row after row of pockets across the film. The pockets are filled with an inventive formulation, and as the pockets move through the dies, they are sealed, shaped, and cut from the moving film as capsules filled with agent formulation. A semipermeable encapsulating lamina is coated thereon to yield the capsule. The continuous process is a manufacturing system that also uses rotary dies, with the added feature that the process can successfully fill active agent in dry powder form into a soft capsule, in addition to encapsulating liquids. The filled capsule of the continuous process is encapsulated with a semipermeable polymeric material to yield the capsule. Procedures for manufacturing soft capsules are disclosed in U.S. Pat. No. 4,627,850 and U.S. Pat. No. 6,419,952.

The dosage forms of the present invention can also be made from an injection-moldable composition by an injection-molding technique. Injection-moldable compositions provided for injection-molding into the semipermeable wall comprise a thermoplastic polymer, or the compositions comprise a mixture of thermoplastic polymers and optional injection-molding ingredients. The thermoplastic polymer that can be used for the present purpose comprise polymers that have a low softening point, for example, below 200° C., preferably within the range of 40° C. to 180° C. The polymers, are preferably synthetic resins, addition polymerized resins, such as polyamides, resins obtained from diepoxides and primary alkanolamines, resins of glycerine and phthalic anhydrides, polymethane, polyvinyl resins, polymer resins with end-positions free or esterified carboxyl or caboxamide groups, for example with acrylic acid, acrylic amide, or acrylic acid esters, polycaprolactone, and its copolymers with dilactide, diglycolide, valerolactone and decalactone, a resin composition comprising polycaprolactone and polyalkylene oxide, and a resin composition comprising polycaprolactone, a polyalkylene oxide such as polyethylene oxide, poly(cellulose) such as poly(hydroxypropylmethylcellulose), poly(hydroxyethylmethylcellulose), and poly(hydroxypropylcellulose). The membrane forming composition can comprise optional membrane-forming ingredients such as polyethylene glycol, talcum, polyvinylalcohol, lactose, or polyvinyl pyrrolidone. The compositions for forming an injection-molding polymer composition can comprise 100% thermoplastic polymer. The composition in another embodiment comprises 10% to 99% of a thermoplastic polymer and 1% to 90% of a different polymer with the total equal to 100%. The invention provides also a thermoplastic polymer composition comprising 1% to 98% of a first thermoplastic polymer, 1% to 90% of a different, second polymer and 1% to 90% of a different, third polymer with all polymers equal to 100%. Representation composition comprises 20% to 90% of thermoplastic polycaprolactone and 10% to 80% of poly(alkylene oxide); a composition comprising 20% to 90% polycaprolactone and 10% to 60% of poly(ethylene oxide) with the ingredients equal to 100%; a composition comprising 10% to 97% of polycaprolactone, 10% to 97% poly(alkylene oxide), and 1% to 97% of poly(ethylene glycol) with all ingredients equal to 100%; a composition comprising 20% to 90% polycaprolactone and 10% to 80% of poly(hydroxypropylcellulose) with all ingredients equal to 100%; and a composition comprising 1% to 90% polycaprolactone, 1% to 90% poly(ethylene oxide), 1% to 90% poly(hydroxypropylcellulose) and 1% to 90% poly(ethylene glycol) with all ingredients equal to 100%. The percent expressed is weight percent wt %.

In another embodiment of the invention, a composition for injection-molding to provide a membrane can be prepared by blending a composition comprising a polycaprolactone 63 wt %, polyethylene oxide 27 wt %, and polyethylene glycol 10 wt % in a conventional mixing machine, such as a Moriyama™ Mixer at 65° C. to 95° C., with the ingredients added to the mixer in the following addition sequence, polycaprolactone, polyethylene oxide and polyethylene glycol. In one example, all the ingredients are mixed for 135 minutes at a rotor speed of 10 to 20 rpm. Next, the blend is fed to a Baker Perkins Kneader™ extruder at 80° C. to 90° C., at a pump speed of 10 rpm and a screw speed of 22 rpm, and then cooled to 10° C. to 12° C., to reach a uniform temperature. Then, the cooled extruded composition is fed to an Albe Pelletizer, converted into pellets at 250° C., and a length of 5 mm. The pellets next are fed into an injection-molding machine, an Arburg Allrounder™ at 200° F. to 350° C. (93° C. to 177° C.), heated to a molten polymeric composition, and the liquid polymer composition forced into a mold cavity at high pressure and speed until the mold is filled and the composition comprising the polymers are solidified into a preselected shape. The parameters for the injection-molding consists of a band temperature through zone 1 to zone 5 of the barrel of 195° F. (91° C.) to 375° F., (191° C.), an injection-molding pressure of 1818 bar, a speed of 55 cm3/s, and a mold temperature of 75° C. The injection-molding compositions and injection-molding procedures are disclosed in U.S. Pat. No. 5,614,578.

Alternatively, the capsule can be made conveniently in two parts, with one part (the “cap”) slipping over and capping the other part (the “body”) as long as the capsule is deformable under the forces exerted by the expandable layer and seals to prevent leakage of the liquid, active agent formulation from between the telescoping portions of the body and cap. The two parts completely surround and capsulate the internal lumen that contains the liquid formulation, which can contain useful additives. The two parts can be fitted together after the body is filled with the liquid formulation. The assembly can be done by slipping or telescoping the cap section over the body section, and sealing the cap and body, thereby completely surrounding and encapsulating the liquid formulation.

Soft capsules typically have a wall thickness that is greater than the wall thickness of hard capsules. For example, soft capsules can, for example, have a wall thickness on the order of 10-40 mils, about 20 mils being typical, whereas hard capsules can, for example, have a wall thickness on the order of 2-6 mils, about 4 mils being typical.

In one embodiment of the dosage system, a soft capsule can be of single unit construction and can be surrounded by an unsymmetrical hydro-activated layer as the expandable layer. The expandable layer will generally be unsymmetrical and have a thicker portion remote from the exit orifice. As the hydro-activated layer imbibes and/or absorbs external fluid, it expands and applies a push pressure against the wall of capsule and optional barrier layer and forces active agent formulation through the exit orifice. The presence of an unsymmetrical layer functions to assure that the maximum dose of agent is delivered from the dosage form, as the thicker section of layer distant from passageway swells and moves towards the orifice.

In yet another configuration, the expandable layer can be formed in discrete sections that do not entirely encompass an optionally barrier layer-coated capsule. The expandable layer can be a single element that is formed to fit the shape of the capsule at the area of contact. The expandable layer can be fabricated conveniently by tableting to form the concave surface that is complementary to the external surface of the barrier-coated capsule. Appropriate tooling such as a convex punch in a conventional tableting press can provide the necessary complementary shape for the expandable layer. In this case, the expandable layer is granulated and compressed, rather than formed as a coating. The methods of formation of an expandable layer by tableting are well known, having been described, for example in U.S. Pat. Nos. 4,915,949; 5,126,142; 5,660,861; 5,633,011; 5,190,765; 5,252,338; 5,620,705; 4,931,285; 5,006,346; 5,024,842; and 5,160,743.

In some embodiments, a barrier layer can be first coated onto the capsule and then the tableted, expandable layer is attached to the barrier-coated capsule with a biologically compatible adhesive. Suitable adhesives include, for example, starch paste, aqueous gelatin solution, aqueous gelatin/glycerin solution, acrylate-vinylacetate based adhesives such as Duro-Tak adhesives (National Starch and Chemical Company), aqueous solutions of water soluble hydrophilic polymers such as hydroxypropyl methyl cellulose, hydroxymethyl cellulose, hydroxyethyl cellulose, and the like. That intermediate dosage form can be then coated with a semipermeable layer. The exit orifice is formed in the side or end of the capsule opposite the expandable layer section. As the expandable layer imbibes fluid, it will swell. Since it is constrained by the semipermeable layer, as it expands it will compress the barrier-coated capsule and express the liquid formulation from the interior of the capsule into the environment of use.

The hard capsules are typically composed of two parts, a cap and a body, which are fitted together after the larger body is filled with a preselected appropriate formulation. This can be done by slipping or telescoping the cap section over the body section, thus completely surrounding and encapsulating the liquid formulation. Hard capsules can be made, for example, by dipping stainless steel molds into a bath containing a solution of a capsule lamina-forming material to coat the mold with the material. Then, the molds are withdrawn, cooled, and dried in a current of air. The capsule is stripped from the mold and trimmed to yield a lamina member with an internal lumen. The engaging cap that telescopically caps the formulation receiving body is made in a similar manner. Then, the closed and filled capsule can be encapsulated with a semipermeable lamina. The semipermeable lamina can be applied to capsule parts before or after parts and are joined into the final capsule. In another embodiment, the hard capsules can be made with each part having matched locking rings near their opened end that permit joining and locking together the overlapping cap and body after filling with formulation. In this embodiment, a pair of matched locking rings are formed into the cap portion and the body portion, and these rings provide the locking means for securely holding together the capsule. The capsule can be manually filled with the formulation, or they can be machine filled with the formulation. In the final manufacture, the hard capsule is encapsulated with a semipermeable lamina permeable to the passage of fluid and substantially impermeable to the passage of benzisoxazole derivative. Methods of forming hard cap dosage forms are described in U.S. Pat. No. 6,174,547, U.S. Pat. Nos. 6,596,314, 6,419,952, and 6,174,547.

The hard and soft capsules can comprise, for example, gelatin; gelatin having a viscosity of 15 to 30 millipoises and a bloom strength up to 150 grams; gelatin having a bloom value of 160 to 250; a composition comprising gelatin, glycerine, water and titanium dioxide; a composition comprising gelatin, erythrosin, iron oxide and titanium dioxide; a composition comprising gelatin, glycerine, sorbitol, potassium sorbate and titanium dioxide; a composition comprising gelatin, acacia glycerine, and water; and the like. Materials useful for forming the capsule wall are known in U.S. Pat. No. 4,627,850; and in U.S. Pat. No. 4,663,148. Alternatively, the capsules can be made out of materials other than gelatin (see for example, products made by BioProgres pic).

The capsules typically can be provided, for example, in sizes from about 3 to about 22 minims (1 minim being equal to 0.0616 ml) and in shapes of oval, oblong or others. They can be provided in standard shape and various standard sizes, conventionally designated as (000), (00), (0), (1), (2), (3), (4), and (5). The largest number corresponds to the smallest size. Non-standard shapes can be used as well. In either case of soft capsule or hard capsule, non-conventional shapes and sizes can be provided if required for a particular application.

The osmotic devices of the present invention may comprise a semipermeable wall permeable to the passage of exterior biological fluid and substantially impermeable to the passage of benzisoxazole derivatives. The selectively permeable compositions used for forming the wall are essentially non-erodible and they are insoluble in biological fluids during the life of the osmotic system. The semipermeable wall comprises a composition that does not adversely affect the host, the liquid formulation, an osmopolymer, osmagent and the like. Materials useful in the formation of a semipermeable wall are disclosed elsewhere herein.

The semipermeable wall can also comprise a flux regulating agent. Materials useful flux regulating agents are disclosed elsewhere herein. Other materials that can be used to form the semipermeable wall for imparting flexibility and elongation properties to the semipermeable wall are also disclosed elsewhere herein.

The semipermeable wall surrounds and forms a compartment containing a one or a plurality of layers, one of which is an expandable layer that in some embodiments, can contain osmotic agents. The composition of such expandable layers is disclosed elsewhere herein.

In certain solid and liquid embodiments, the dosage forms further can comprise a barrier layer. The barrier layer in certain embodiments is deformable under the pressure exerted by the expandable layer and will be impermeable (or less permeable) to fluids and materials that can be present in the expandable layer, the liquid formulation and in the environment of use, during delivery of the liquid formulation. A certain degree of permeability of the barrier layer can be permitted if the delivery rate of the liquid formulation is not detrimentally effected. However, it is preferred that barrier layer not completely transport through it fluids and materials in the dosage form and the environment of use during the period of delivery of the liquid formulation. The barrier layer can be deformable under forces applied by expandable layer so as to permit compression of capsule to force the liquid formulation from the exit orifice. In some embodiments, the barrier layer will be deformable to such an extent that it create a seal between the expandable layer and the semipermeable layer in the area where the exit orifice is formed. In that manner, the barrier layer will deform or flow to a limited extent to seal the initially, exposed areas of the expandable layer and the semipermeable layer when the exit orifice is being formed, such as by drilling or the like, or during the initial stages of operation. When sealed, the only avenue for liquid permeation into the expandable layer is through the semipermeable layer, and there is no back-flow of fluid into the expandable layer through the exit orifice.

Suitable materials for forming the barrier layer can include, for example, polyethylene, polystyrene, ethylene-vinyl acetate copolymers, polycaprolactone and Hytrel™ polyester elastomers (Du Pont), cellulose acetate, cellulose acetate pseudolatex (such as described in U.S. Pat. No. 5,024,842), cellulose acetate propionate, cellulose acetate butyrate, ethyl cellulose, ethyl cellulose pseudolatex (such as Surelease™ as supplied by 10 Colorcon, West Point, Pa. or Aquacoat™ as supplied by FMC Corporation, Philadelphia, Pa.), nitrocellulose, polylactic acid, poly-glycolic acid, polylactide glycolide copolymers, collagen, polyvinyl alcohol, polyvinyl acetate, polyethylene vinylacetate, polyethylene teraphthalate, polybutadiene styrene, polyisobutylene, polyisobutylene isoprene copolymer, polyvinyl chloride, polyvinylidene chloride-vinyl chloride copolymer, copolymers of acrylic acid and methacrylic acid esters, copolymers of methylmethacrylate and ethylacrylate, latex of acrylate esters (such as Eudragit™ supplied by RohmPharma, Darmstaat, Germany), polypropylene, copolymers of propylene oxide and ethylene oxide, propylene oxide ethylene oxide block copolymers, ethylenevinyl alcohol copolymer, polysulfone, ethylene vinylalcohol copolymer, polyxylylenes, polyalkoxysilanes, polydimethyl siloxane, polyethylene glycol-silicone elastomers, electromagnetic irradiation crosslinked acrylics, silicones, or polyesters, thermally crosslinked acrylics, silicones, or polyesters, butadiene-styrene rubber, and blends of the above.

Preferred materials can include cellulose acetate, copolymers of acrylic acid and methacrylic acid esters, copolymers of methylmethacrylate and ethylacrylate, and latex of acrylate esters. Preferred copolymers can include poly (butyl methacrylate), (2-dimethylaminoethyl)methacrylate, methyl methacrylate) 1:2:1, 150,000, sold under the trademark EUDRAGIT E; poly (ethyl acrylate, methyl methacrylate) 2:1, 800,000, sold under the trademark EUDRAGIT NE 30 D; poly (methacrylic acid, methyl methacrylate) 1:1, 135,000, sold under the trademark EUDRAGIT L; poly (methacrylic acid, ethyl acrylate) 1:1, 250,000, sold under the trademark EUDRAGIT L; poly (methacrylic acid, methyl methacrylate) 1:2, 135,000, sold under the trademark EUDRAGIT S; poly (ethyl acrylate, methyl methacrylate, trimethylammonioethyl methacrylate chloride) 1:2:0.2, 150,000, sold under the trademark EUDRAGIT RL; poly (ethyl acrylate, methyl methacrylate, trimethylammonioethyl methacrylate chloride) 1:2:0.1, 150,000, sold as EUDRAGIT RS. In each case, the ratio x:y:z indicates the molar proportions of the monomer units and the last number is the number average molecular weight of the polymer. Especially preferred are cellulose acetate containing plasticizers such as acetyl tributyl citrate and ethylacrylate methylmethylacrylate copolymers such as Eudragit NE.

The foregoing materials for use as the barrier layer can be formulated with plasticizers to make the barrier layer suitably deformable such that the force exerted by the expandable layer will collapse the compartment formed by the barrier layer to dispense the liquid formulation. Examples of typical plasticizers are as follows: polyhydric alcohols, triacetin, polyethylene glycol, glycerol, propylene glycol, acetate esters, glycerol triacetate, triethyl citrate, acetyl triethyl citrate, glycerides, acetylated monoglycerides, oils, mineral oil, castor oil and the like. The plasticizers can be blended into the material in amounts of 10-50 weight percent based on the weight of the material.

The various layers forming the barrier layer, expandable layer and semipermeable layer can be applied by conventional coating methods such as described in U.S. Pat. No. 5,324,280. While the barrier layer, expandable layer and semipermeable wall have been illustrated and described for convenience as single layers, each of those layers can be composites of several layers. For example, for particular applications it may be desirable to coat the capsule with a first layer of material that facilitates coating of a second layer having the permeability characteristics of the barrier layer. In that instance, the first and second layers comprise the barrier layer. Similar considerations would apply to the semipermeable layer and the expandable layer.

The exit orifice can be formed by mechanical drilling, laser drilling, eroding an erodible element, extracting, dissolving, bursting, or leaching a passageway former from the composite wall. The exit orifice can be a pore formed by leaching sorbitol, lactose or the like from a wall or layer as disclosed in U.S. Pat. No. 4,200,098. This patent discloses pores of controlled-size porosity formed by dissolving, extracting, or leaching a material from a wall, such as sorbitol from cellulose acetate. A preferred form of laser drilling is the use of a pulsed laser that incrementally removes material from the composite wall to the desired depth to form the exit orifice.

### EXAMPLES

### Example 1

### 2 mg Risperidone Osmotic Module Formulation with Polysorbate 80

First, a push composition was prepared as follows: first, a binder solution was prepared. 4.3 kg of hydroxypropyl methylcellulose identified as 2910 was dissolved in 38.7 kg of water. Then, 36 kg of sodium chloride and 0.36 kg of ferric oxide were sized using a Quadro Comil with a 21-mesh screen. Then, the screened materials, 2.4 kg of hydroxypropyl methylcellulose identified as 2910 and 76.44 kg of polyethylene oxide (approximately 7,000,000 molecular weight) were added to a fluid bed granulator bowl. The dry materials were fluidized and mixed while 36 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 60 g of butylated hydroxytoluene and lubricated with 1.14 kg of stearic acid.

Next, the barrier layer was prepared as follows: 3 kg of polyvinyl acetate/povidone and 3 kg of microfine wax, grade MF-2JH were charged to the bowl of the Hobart mixer. The dry components were mixed for 5 minutes. Then, water was added to the mixing bowl at a constant rate to reach acceptable granulation results. The resulting wet granulation was manually pressed through a 16-mesh screen and dried at 50 Deg C. to an acceptable moisture level. Finally, the dry granulation was manually sized using a 16-mesh screen

Next, the push and the barrier layer granulations were compressed into bilayer arrangements. 85 mg of barrier layer granulation was compressed with 270 mg of push layer granulation using the rotary tablet press with 0.278″ (7 mm) tooling.

Next, the osmotic module was assembled as follows: bilayer arrangements of push and barrier layers were inserted to a depth of 0.525 inches into the size O, transparent HPMC capsule body.

Next, the assembled osmotic modules were coated with a semi-permeable wall. The wall forming composition comprised 90% cellulose acetate having a 39.8% acetyl content and 10% poloxamer 188. The wall-forming composition was dissolved in acetone. The wall-forming composition was sprayed onto and around the bilayered arrangements in a pan coater until approximately 60 mg of membrane was applied to each tablet.

Next, a 20 mil (0.51 mm)) exit passageway was drilled through the semi-permeable wall to connect the drug layer with the exterior of the dosage system. The residual solvent was removed by drying at 45° C. and 45% RH for 24 hours followed by drying at 45° C. and ambient humidity for additional 24 hours.

Next, a liquid drug layer composition was prepared as follows: 29.862 g of polysorbate 80 was weighed into the glass jar. Then, 15 mg of butylated hydroxytoluene was mixed with polysorbate 80 for 30 seconds. Finally, 0.123 g of risperidone was added into solution, pre-mixed with a spatula for 30 seconds and then mixed on a stirring plate for 20 hours.

Next, the empty compartment of the osmotic module was filled with a liquid drug layer using syringe. Approximately 500 mg of the liquid drug layer was dispensed into each osmotic module.

### Example 2

### 2 mg Paliperidone Osmotic Module Formulation with Polysorbate 80

First, a push composition was prepared as follows: first, a binder solution was prepared. 4.3 kg of hydroxypropyl methylcellulose identified as 2910 was dissolved in 38.7 kg of water. Then, 36 kg of sodium chloride and 0.36 kg of ferric oxide were sized using a Quadro Comil with a 21-mesh screen. Then, the screened materials, 2.4 kg of hydroxypropyl methylcellulose identified as 2910 and 76.44 kg of polyethylene oxide (approximately 7,000,000 molecular weight) were added to a fluid bed granulator bowl. The dry materials were fluidized and mixed while 36 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 60 g of butylated hydroxytoluene and lubricated with 1.14 kg of stearic acid.

Next, the barrier layer was prepared as follows: 3 kg of polyvinyl acetate/povidone and 3 kg of microfine wax, grade MF-2JH were charged to the bowl of the Hobart mixer. The dry components were mixed for 5 minutes. Then, water was added to the mixing bowl at a constant rate to reach acceptable granulation results. The resulting wet granulation was manually pressed through a 16-mesh screen and dried at 50 Deg C. to an acceptable moisture level. Finally, the dry granulation was manually sized using a 16-mesh screen

Next, the push and the barrier layer granulations were compressed into bilayer arrangements. 85 mg of barrier layer granulation was compressed with 270 mg of push layer granulation using the rotary tablet press with 0.278″ (7 mm) tooling.

Next, the osmotic module was assembled as follows: bilayer arrangements of push and barrier layers were inserted to a depth of 0.525 inches into the size O, transparent HPMC capsule body.

Next, the assembled osmotic modules were coated with a semi-permeable wall. The wall forming composition comprised 90% cellulose acetate having a 39.8% acetyl content and 10% poloxamer 188. The wall-forming composition was dissolved in acetone. The wall-forming composition was sprayed onto and around the bilayered arrangements in a pan coater until approximately 60 mg of membrane was applied to each tablet.

Next, a 20 mil (0.51 mm)) exit passageway was drilled through the semi-permeable wall to connect the drug layer with the exterior of the dosage system. The residual solvent was removed by drying at 45° C. and 45% RH for 24 hours followed by drying at 45° C. and ambient humidity for additional 24 hours.

Next, a liquid drug layer composition was prepared as follows: 29.862 g of polysorbate 80 was weighed into the glass jar. Then, 15 mg of butylated hydroxytoluene was mixed with polysorbate 80 for 30 seconds. Finally, 0.123 g of paliperidone was added into solution, pre-mixed with a spatula for 30 seconds and then mixed on a stirring plate for 20 hours.

Next, the empty compartment of the osmotic module was filled with a liquid drug layer using syringe. Approximately 500 mg of the liquid drug layer was dispensed into each osmotic module.

Next, the empty compartment of the osmotic module was filled with liquid drug layer using syringe. Approximately 500 mg of the liquid drug layer was dispensed into each osmotic module.

### Example 3

### 2 mg Risperidone Osmotic Module Formulation with Cremophor

First, a push composition was prepared as follows: first, a binder solution was prepared. 4.3 kg of hydroxypropyl methylcellulose identified as 2910 was dissolved in 38.7 kg of water. Then, 36 kg of sodium chloride and 0.36 kg of ferric oxide were sized using a Quadro Comil with a 21-mesh screen. Then, the screened materials, 2.4 kg of hydroxypropyl methylcellulose identified as 2910 and 76.44 kg of Polyethylene oxide (approximately 7,000,000 molecular weight) were added to a fluid bed granulator bowl. The dry materials were fluidized and mixed while 36 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 60 g of butylated hydroxytoluene and lubricated with 1.14 kg of stearic acid.

Next, the barrier layer was prepared as follows: 3 kg of polyvinyl acetate/povidone and 3 kg of microfine wax, grade MF-2JH were charged to the bowl of the Hobart mixer. The dry components were mixed for 5 minutes. Then, water was added to the mixing bowl at a constant rate to reach acceptable granulation results. The resulting wet granulation was manually pressed through a 16-mesh screen and dried at 50 Deg C. to an acceptable moisture level. Finally, the dry granulation was manually sized using a 16-mesh screen

Next, the push and the barrier layer granulations were compressed into bilayer arrangements. 85 mg of barrier layer granulation was compressed with 270 mg of push layer granulation using the rotary tablet press with 0.278″ (7 mm) tooling.

Next, the osmotic module was assembled as follows: bilayer arrangements of push and barrier layers were inserted to a depth of 0.525 inches into the size O, transparent HPMC capsule body.

Next, the assembled osmotic modules were coated with a semi-permeable wall. The wall forming composition comprised 90% cellulose acetate having a 39.8% acetyl content and 10% poloxamer 188. The wall-forming composition was dissolved in acetone. The wall-forming composition was sprayed onto and around the bilayered arrangements in a pan coater until approximately 60 mg of membrane was applied to each tablet.

Next, a 20 mil (0.51 mm)) exit passageway was drilled through the semi-permeable wall to connect the drug layer with the exterior of the dosage system. The residual solvent was removed by drying at 45° C. and 45% RH for 24 hours followed by drying at 45° C. and ambient humidity for additional 24 hours.

Next, a liquid formulation was prepared as follows: 29.862 g of ethoxylated castor oil (Cremophor EL) was weighed into the glass jar. Then, 15 mg of butylated hydroxytoluene was mixed with polysorbate 80 for 30 seconds. Finally, 0.123 g of risperidone was added into solution, pre-mixed with a spatula for 30 seconds and then mixed on a stirring plate for 20 hours.

Next, the empty compartment of the osmotic module was filled with a liquid formulation using a syringe. Approximately 500 mg of the liquid formulation was dispensed into each osmotic module. The exit passageway was left unplugged.

### Example 4

### 2 mg Risperidone Osmotic Module Formulation with Poloxamer L-44

First, a push composition was prepared as follows: first, a binder solution was prepared. 4.3 kg of hydroxypropyl methylcellulose identified as 2910 was dissolved in 38.7 kg of water. Then, 36 kg of sodium chloride and 0.36 kg of ferric oxide were sized using a Quadro Comil with a 21-mesh screen. Then, the screened materials, 2.4 kg of hydroxypropyl methylcellulose identified as 2910 and 76.44 kg of Polyethylene oxide (approximately 7,000,000 molecular weight) were added to a fluid bed granulator bowl. The dry materials were fluidized and mixed while 36 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 60 g of butylated hydroxytoluene and lubricated with 1.14 kg of stearic acid.

Next, the barrier layer was prepared as follows: 3 kg of polyvinyl acetate/povidone and 3 kg of microfine wax, grade MF-2JH were charged to the bowl of the Hobart mixer. The dry components were mixed for 5 minutes. Then, water was added to the mixing bowl at a constant rate to reach acceptable granulation results. The resulting wet granulation was manually pressed through a 16-mesh screen and dried at 50 Deg C. to an acceptable moisture level. Finally, the dry granulation was manually sized using a 16-mesh screen

Next, the push and the barrier layer granulations were compressed into bilayer arrangements. 85 mg of barrier layer granulation was compressed with 270 mg of push layer granulation using the rotary tablet press with 0.278″ (7 mm) tooling.

Next, the osmotic module was assembled as follows: bilayer arrangements of push and barrier layers were inserted to a depth of 0.525 inches into the size O, transparent HPMC capsule body.

Next, the assembled osmotic modules were coated with a semi-permeable wall. The wall forming composition comprised 90% cellulose acetate having a 39.8% acetyl content and 10% poloxamer 188. The wall-forming composition was dissolved in acetone. The wall-forming composition was sprayed onto and around the bilayered arrangements in a pan coater until approximately 60 mg of membrane was applied to each tablet.

Next, a 20 mil (0.51 mm)) exit passageway was drilled through the semi-permeable wall to connect the drug layer with the exterior of the dosage system. The residual solvent was removed by drying at 45° C. and 45% RH for 24 hours followed by drying at 45° C. and ambient humidity for additional 24 hours.

Next, a liquid formulation was prepared as follows: 29.862 g of polyoxyethylene-polyoxypropylene copolymer (Poloxamer L-44) was weighed into the glass jar. Then, 15 mg of butylated hydroxytoluene was mixed with polysorbate 80 for 30 seconds. Finally, 0.123 g of risperidone was added into solution, pre-mixed with a spatula for 30 seconds and then mixed on a stirring plate for 20 hours.

Next, the empty compartment of the osmotic module was filled with a liquid formulation using a syringe. Approximately 500 mg of the liquid formulation was dispensed into each osmotic module. The exit passageway was left unplugged.

### Example 5

### In Vitro Release Rate Testing

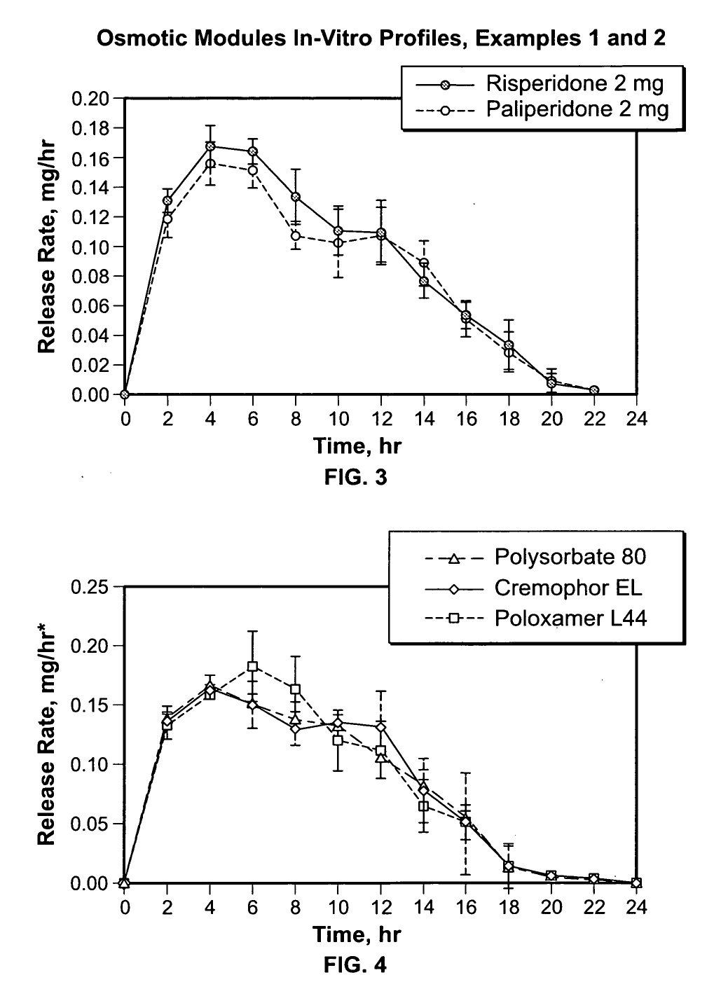

Dosage forms produced according to Examples 1 and 2 were tested to determine the paliperidone and risperidone release rate, as appropriate, using the test methods generally laid out as follows. The results are shown in FIG. 3

Paliperidone Release Rate Test Method

The high performance liquid chromatography (HPLC) method employs USP Type VII Release Rate Apparatus. Samples were released into 50 mL of modified AGF. Aliquots of the release rate sample solutions were injected into a chromatographic system to quantify the amounts of drug released during specified test intervals. Paliperidone was resolved on a C18 column and detected by UV absorption at 275 nm. Quantitation of paliperidone was performed by linear regression analysis of peak areas from a standard curve containing at least five standard points.

Supplies used were: Calibrated release rate tube 50 mL, USP Type VII Release Rate Apparatus, Class A volumetric flasks (25, 50, 100 and 200 mL), Class A volumetric pipettes (2, 5 and 15 mL), Kontes Ultra Ware Filtration system, or equivalent, Eppendorf Centrifuge 5415C, or equivalent, Traceable VWR DigitalThermometer, or equivalent, Beckman 260 pH meter, or equivalent, Analytical balance, Mettler Toledo, or equivalent, Variable-speed laboratory stirrer, Magnetic stirring bars (size of stirring bars suitable for flask size), Graduated cylinder (1000 mL), Methanol (MeOH), HPLC grade, Acetonitrile (ACN), HPLC grade, Milli-Q grade water, Paliperidone reference standard, Formic acid GR, A.C.S. reagent or equivalent, Formic acid ammonium salt, A.C.S. reagent or equivalent, Hydrochloric Acid 5N solution, A.C.S. reagent, or equivalent, Sodium chloride crystal, A.C.S reagent, or equivalent.

The HPLC Mobile Phase was prepared as follows. First, for the 0.05 M Ammonium Formate Buffer, pH 3.3±0.1 approximately 4.2 g of formic acid ammonium salt was weight and transferred to a 4 L flask. 2 liters of water was added and mixed until the salt is dissolved. Then, 5 mL of formic acid was added and mixed and a final volume of 4 L was achieved with water. Then, 3000 mL of 0.05M ammonium formate buffer, 320 mL of ACN, 680 mL of methanol were measured individually, combined and mixed well. The solution was filtered prior to use.

The standard diluting solvent was prepared by combining and mixing 250 mL of methanol with 750 mL of water.

Release Rate Media (Modified AGF), pH 1.0±0.5 was prepared as follows: approximately 8 g of sodium chloride was weighed and transfered into a 4 L flask. Two liters of water was added and mixed. Then, 66 mL of 5N HCl solution and 1934 mL of water were added and mixed.

The HPLC operating parameters were set as follows: flow rate—1.5 mL/min, detector wavelength—275 nm, temperature—35 deg.C, run time—5.5 min

The paliperidone stock solution was prepared by weighting approximately 20.0 mg of paliperidone reference standard into a 200 mL volumetric flask; the drug was rinsed into the flask using methanol, swirled to dissolve and diluted to final volume with methanol

Next, each dosage form that was to be tested was weighed and the weight recorded. Each dosage form was placed in a prong sample holder. The prong sample holder was attached to the USP VII bath indexer that operated at vertical reciprocating amplitude of about 2-3 cm, and a frequency of about 30 cycles per minute. The dosage forms were released into 50 mL calibrated test tubes containing 50 mL of the release media at 37.0° C.±0.5° C. such that the dosage forms were continuously immersed. Test tube solutions were pre-equilibrated in a constant temperature water bath controlled to 37.0° C.±0.5° C.

At the end of each two-hour test interval, the dosage forms were transferred to the next row of test tubes containing fresh release media. After release, the tubes were removed from the bath and allowed to cool to ambient temperature. The release solution in each tube was brought up to the 50 ml mark with release media, and thoroughly mixed 30 times using an inert stirring rod fitted with a disk perpendicular to the rod. Sample solutions were centrifuged at room temperature for about 10 minutes at approximately 10,000 rpm or until solution is clear. An aliquot was transferred to an HPLC vial.

A system suitability test was performed by equilibrating the HPLC system until a steady baseline is obtained and injecting mid-level working standard solution five times. The system was considered suitable for analysis if the following minimum chromatographic performance requirements were met:

Capacity Factor (k′)≧1.5

Tailing Factor (T) T≦2.5

Area Response Variation RSD≦2.0%

Retention Time Variation RSD≦2.0%

Adjustments were made to run time or columns were replaced as necessary to obtain optimum performance.

Blank solutions, paliperidone calibration working standards and QC working standard were injected prior to sample analysis. Then, the samples were injected with periodical standard checks (every 24 injections).

A calibration curve of peak areas versus concentrations of working standards was constructed. The concentration of paliperidone in the samples was determined from a linear regression analysis (LRA) of the calibration curve. The results were calculated as follows:

$$\frac{mg}{hour} ( paliperidone ) = \frac{C \times V}{T}$$

$$Cumulative mg ( paliperidone ) = ( {C}_{1} + {C}_{2} + … + {C}_{n} ) \times V$$

where:
V=Volume of release media, 50 mL
C=Drug concentration at specified time interval as determined by LRA or calibration curve in mg/mL
C₁=Drug concentration at first specified interval
C₂=Drug concentration at second specified interval
Cₙ=Drug concentration at final interval
T=Time interval (2 hr)
   Risperidone Release Rate Test Method

Dosage forms were tested to determine the risperidone release rate by high performance liquid chromatography (HPLC). The method employs the USP Type VII Release Rate Apparatus. Samples were released into 50 mL of modified AGF. Aliquots of the release rate sample solutions were injected into a chromatographic system to quantify the amounts of drug released during specified test intervals. Risperidone was resolved on a C18 column and detected by UV absorption at 275 nm. Quantitation of risperidone was performed by linear regression analysis of peak areas from a standard curve containing at least five standard points.

Supplies used were: Calibrated release rate tube 50 mL, USP Type VII Release Rate Apparatus, Class A volumetric flasks (25, 50, 100 and 200 mL), Class A volumetric pipettes (2, 5 and 15 mL), Kontes Ultra Ware Filtration system, or equivalent, Eppendorf Centrifuge 5415C, or equivalent, Traceable VWR DigitalThermometer, or equivalent, Beckman 260 pH meter, or equivalent, Analytical balance, Mettler Toledo, or equivalent, Variable-speed laboratory stirrer, Magnetic stirring bars (size of stirring bars suitable for flask size), Graduated cylinder (1000 mL), Methanol (MeOH), HPLC grade, Acetonitrile (ACN), HPLC grade, Milli-Q grade water, Risperidone reference standard, Formic acid GR, A.C.S. reagent or equivalent, Formic acid ammonium salt, A.C.S. reagent or equivalent, Hydrochloric Acid 5N solution, A.C.S. reagent, or equivalent, Sodium chloride crystal, A.C.S reagent, or equivalent.

The HPLC Mobile Phase was prepared as follows. First, for the 0.05 M Ammonium Formate Buffer, pH 3.3±0.1 approximately 4.2 g of formic acid ammonium salt was weight and transferred to a 4 L flask. 2 liters of water was added and mixed until the salt is dissolved. Then, 5 mL of formic acid was added and mixed and a final volume of 4 L was achieved with water. Then, 3000 mL of 0.05M ammonium formate buffer, 320 mL of ACN, 680 mL of methanol were measured individually, combined and mixed well. The solution was filtered prior to use.

The standard diluting solvent was prepared by combining and mixing 250 mL of methanol with 750 mL of water.

Release Rate Media (Modified AGF), pH 1.0±0.5 was prepared as follows: approximately 8 g of sodium chloride was weighed and transfered into a 4 L flask. Two liters of water was added and mixed. Then, 66 mL of 5N HCl solution and 1934 mL of water were added and mixed.

The HPLC operating parameters were set as follows: flow rate—1.5 mL/min, detector wavelength—275 nm, temperature—35 deg.C, run time—5.5 min

The risperidone stock solution was prepared by weighting approximately 20.0 mg of risperidone reference standard into a 200 mL volumetric flask; the drug was rinsed into the flask using methanol, swirled to dissolve and diluted to final volume with methanol

Next, each dosage form that was to be tested was weighed and the weight recorded. Each dosage form was placed in a prong sample holder. The prong sample holder was attached to the USP VII bath indexer that operated at vertical reciprocating amplitude of about 2-3 cm, and a frequency of about 30 cycles per minute. The dosage forms were released into 50 mL calibrated test tubes containing 50 mL of the release media at 37.0° C.±0.5° C. such that the dosage forms were continuously immersed. Test tube solutions were pre-equilibrated in a constant temperature water bath controlled to 37.0° C.±0.5° C.

At the end of each two-hour test interval, the dosage forms were transferred to the next row of test tubes containing fresh release media. After release, the tubes were removed from the bath and allowed to cool to ambient temperature. The release solution in each tube was brought up to the 50 ml mark with release media, and thoroughly mixed 30 times using an inert stirring rod fitted with a disk perpendicular to the rod. Sample solutions were centrifuged at room temperature for about 10 minutes at approximately 10,000 rpm or until solution is clear. An aliquot was transferred to an HPLC vial.

A system suitability test was performed by equilibrating the HPLC system until a steady baseline is obtained and injecting mid-level working standard solution five times. The system was considered suitable for analysis if the following minimum chromatographic performance requirements were met:
Capacity Factor (k′)≧1.5
Tailing Factor (T) T≦2.5
Area Response Variation RSD≦2.0%
Retention Time Variation RSD≦2.0%

Adjustments were made to run time or columns were replaced as necessary to obtain optimum performance.

Blank solutions, risperidone calibration working standards and QC working standard were injected prior to sample analysis. Then, the samples were injected with periodical standard checks (every 24 injections).

A calibration curve of peak areas versus concentrations of working standards was constructed. The concentration of risperidone in the samples was determined from a linear regression analysis (LRA) of the calibration curve. The results were calculated as follows:

$$\frac{mg}{hour} ( paliperidone ) = \frac{C \times V}{T}$$

$$Cumulative mg ( paliperidone ) = ( {C}_{1} + {C}_{2} + … + {C}_{n} ) \times V$$

where:
V=Volume of release media, 50 mL
C=Drug concentration at specified time interval as determined by LRA or calibration curve in mg/mL
C₁=Drug concentration at first specified interval
C₂=Drug concentration at second specified interval
Cₙ=Drug concentration at final interval
T=Time interval (2 hr)

### Example 6

### In Vitro Release Rate Testing

Dosage forms produced according to Examples 1, 3 and 4 were tested to determine the risperidone release rate using the test methods generally laid out in Example 5. The results are shown in FIG. 4

### Example 7

### Paliperidone Capsule Shaped Tablet, Trilayer 2 mg System (“Slow”)

A dosage form adapted, designed and shaped as an osmotic drug delivery device was manufactured as follows: 120 g of paliperidone, 7325 g of polyethylene oxide with average molecular weight of 200,000, and 2000 g of sodium chloride, USP were added to a fluid bed granulator bowl. Next a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 50 g of stearic acid.

Next, a second drug compartment composition was prepared as follows: 280 g of paliperidone and 9165 g of polyethylene oxide with average molecular weight of 200,000 were added to a fluid bed granulator bowl. Next a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 50 g of stearic acid.

Next, a push composition was prepared as follows: first, a binder solution was prepared. 15.6 kg of polyvinylpyrrolidone identified as K29-32 having an average molecular weight of 40,000 was dissolved in 104.4 kg of water. Then, 24 kg of sodium chloride and 1.2 kg of ferric oxide were sized using a Quadro Comil with a 21-mesh screen. Then, the screened materials and 88.44 kg of Polyethylene oxide (approximately 7,000,000 molecular weight) were added to a fluid bed granulator bowl. The dry materials were fluidized and mixed while 46.2 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 15 g of butylated hydroxytoluene and lubricated with 294 g magnesium stearate.

Next, the paliperidone drug compositions for the first and the second compartments and the push composition were compressed into trilayer tablets. First, 50 mg of the paliperidone compartment one composition was added to the die cavity and pre-compressed, then 50 mg of the paliperidone compartment two composition was added to the die cavity and pre-compressed, then 100 mg of the push composition was added and the layers were pressed into a 3/16″ diameter longitudinal, deep concave, trilayer arrangement.

The trilayered arrangements were coated with a subcoat laminate. The wall forming composition comprised 70% hydroxypropyl cellulose identified as EF, having an average molecular weight of 80,000 and 30% of polyvinylpyrrolidone identified as K29-32 having an average molecular weight of 40,000. The wall-forming composition was dissolved in anhydrous ethyl alcohol, to make an 8% solids solution. The wall-forming composition was sprayed onto and around the bilayered arrangements in a pan coater until approximately 20 mg of laminate was applied to each tablet.

The trilayered arrangements were coated with a semi-permeable wall. The wall forming composition comprised 99% cellulose acetate having a 39.8% acetyl content and 1% polyethylene glycol comprising a 3.350 viscosity-average molecular weight. The wall-forming composition was dissolved in an acetone:water (95:5 wt:wt) co solvent to make a 5% solids solution. The wall-forming composition was sprayed onto and around the bilayered arrangements in a pan coater until approximately 40 mg of membrane was applied to each tablet.

Next, two 25 mil (0.6 mm) exit passageways were laser drilled through the semi-permeable wall to connect the drug layer with the exterior of the dosage system. The residual solvent was removed by drying for 144 hours as 45 Deg C. and 45% humidity. After drilling, the osmotic systems were dried for 4 hours at 45 Deg C. to remove excess moisture.

The dosage form produced by this manufacture was designed to deliver 2 mg of paliperidone in an ascending delivery pattern from two drug-containing cores. The first core contained 1.2% paliperidone, 73.25% polyethylene oxide possessing a 200,000 molecular weight, 20% sodium chloride, USP, 5% hydroxypropylmethyl cellulose having an average viscosity of 5 cps, 0.05% butylated hydroxytoluene, and 0.5% stearic acid. The second drug core contained 2.8% paliperidone, 91.65% polyethylene oxide possessing a 200,000 molecular weight, 5% hydroxypropylmethyl cellulose having an average viscosity of 5 cps, 0.05% butylated hydroxytoluene, and 0.5% stearic acid. The push composition comprised 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The semi permeable wall was comprised of 99% cellulose acetate of 39.8% acetyl content and 1% polyethylene glycol. The dosage form comprised two passageways, 25 mils (0.6 mm) on the center of the drug side.

### Example 8

### Paliperidone Capsule Shaped Tablet, Trilayer 2 mg System (“Fast”)

A dosage form adapted, designed and shaped as an osmotic drug delivery device was manufactured as follows: 120 g of paliperidone, 7325 g of polyethylene oxide with average molecular weight of 200,000, and 2000 g of sodium chloride, USP were added to a fluid bed granulator bowl. Next a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 50 g of stearic acid.

Next, a second drug compartment composition was prepared as follows: 280 g of paliperidone and 9165 g of polyethylene oxide with an average molecular weight of 200,000 were added to a fluid bed granulator bowl. Next a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 50 g of stearic acid.

Next, a push composition was prepared as follows: first, a binder solution was prepared. 15.6 kg of polyvinylpyrrolidone identified as K29-32 having an average molecular weight of 40,000 was dissolved in 104.4 kg of water. Then, 24 kg of sodium chloride and 1.2 kg of ferric oxide were sized using a Quadro Comil with a 21-mesh screen. Then, the screened materials and 88.44 kg of Polyethylene oxide (approximately 7,000,000 molecular weight) were added to a fluid bed granulator bowl. The dry materials were fluidized and mixed while 46.2 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 15 g of butylated hydroxytoluene and lubricated with 294 g magnesium stearate.

Next, the paliperidone drug compositions for the first and the second compartments and the push composition were compressed into trilayer tablets. First, 50 mg of the paliperidone compartment one composition was added to the die cavity and pre-compressed, then 50 mg of the paliperidone compartment two composition was added to the die cavity and pre-compressed, then 100 mg of the push composition was added and the layers were pressed into a 3/16″ diameter longitudinal, deep concave, trilayer arrangement.

The trilayered arrangements were coated with a subcoat laminate. The wall forming composition comprised 70% hydroxypropyl cellulose identified as EF, having an average molecular weight of 80,000 and 30% of polyvinylpyrrolidone identified as K29-32 having an average molecular weight of 40,000. The wall-forming composition was dissolved in anhydrous ethyl alcohol, to make an 8% solids solution. The wall-forming composition was sprayed onto and around the bilayered arrangements in a pan coater until approximately 20 mg of laminate was applied to each tablet.

The trilayered arrangements were coated with a semi-permeable wall. The wall forming composition comprises 99% cellulose acetate having a 39.8% acetyl content and 1% polyethylene glycol comprising a 3.350 viscosity-average molecular weight. The wall-forming composition was dissolved in an acetone:water (95:5 wt:wt) co solvent to make a 5% solids solution. The wall-forming composition was sprayed onto and around the bilayered arrangements in a pan coater until approximately 20 mg of membrane was applied to each tablet.

Next, two 25 mil (0.6 mm) exit passageways were laser drilled through the semi-permeable wall to connect the drug layer with the exterior of the dosage system. The residual solvent was removed by drying for 144 hours as 45 Deg C. and 45% humidity. After drilling, the osmotic systems were dried for 4 hours at 45 Deg C. to remove excess moisture.

The dosage form produced by this manufacture was designed to deliver 2 mg of paliperidone in an ascending delivery pattern from two drug-containing cores. The first core contained 1.2% paliperidone, 73.25% polyethylene oxide possessing a 200,000 molecular weight, 20% sodium chloride, USP, 5% hydroxypropylmethyl cellulose having an average viscosity of 5 cps, 0.05% butylated hydroxytoluene, and 0.5% stearic acid. The second drug core contained 2.8% paliperidone, 91.65% polyethylene oxide possessing a 200,000 molecular weight, 5% hydroxypropylmethyl cellulose having an average viscosity of 5 cps, 0.05% butylated hydroxytoluene, and 0.5% stearic acid. The push composition comprised 73.7% polyethylene oxide comprising a 7,000,000 molecular weight, 20% sodium chloride, 5% polyvinylpyrrolidone possessing an average molecular weight of 40,000,1% ferric oxide, 0.05% butylated hydroxytoluene, and 0.25% magnesium stearate. The semi permeable wall was comprised of 99% cellulose acetate of 39.8% acetyl content and 1% polyethylene glycol. The dosage form comprised two passageways, 25 mils (0.6 mm) on the center of the drug side.

### Example 9

### Risperidone Capsule Shaped Tablet, Trilayer 2 mg System, FAST

A dosage form adapted, designed and shaped as an osmotic drug delivery device was manufactured as follows: 130 g of risperidone, 7265 g of polyethylene oxide with average molecular weight of 200,000 (super fine particle size), and 2000 g of sodium chloride, USP were added to a fluid bed granulator bowl. Next a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 100 g of stearic acid.

Next, a second drug compartment composition was prepared as follows: 310 g of paliperidone and 9085 g of polyethylene oxide with average molecular weight of 200,000 (super fine particle size) were added to a fluid bed granulator bowl. Next, a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 100 g of stearic acid.

Next, a push composition was prepared as follows: first, a binder solution was prepared. 15.6 kg of polyvinylpyrrolidone identified as K29-32 having an average molecular weight of 40,000 was dissolved in 104.4 kg of water. Then, 24 kg of sodium chloride and 1.2 kg of ferric oxide were sized using a Quadro Comil with a 21-mesh screen. Then, the screened materials and 88.44 kg of Polyethylene oxide (approximately 7,000,000 molecular weight) were added to a fluid bed granulator bowl. The dry materials were fluidized and mixed while 46.2 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 15 g of butylated hydroxytoluene and lubricated with 294 g magnesium stearate.

Next, the paliperidone drug compositions for the first and the second compartments and the push composition were compressed into trilayer tablets. First, 50 mg of the paliperidone compartment one composition was added to the die cavity and pre-compressed, then 40 mg of the paliperidone compartment two composition was added to the die cavity and pre-compressed, then 110 mg of the push composition was added and the layers were pressed into a 3/16″ diameter longitudinal, deep concave, trilayer arrangement.

The trilayered arrangements were coated with a subcoat laminate. The wall forming composition comprised 70% hydroxypropyl cellulose identified as EF, having an average molecular weight of 80,000 and 30% of polyvinylpyrrolidone identified as K29-32 having an average molecular weight of 40,000. The wall-forming composition was dissolved in anhydrous ethyl alcohol, to make an 8% solids solution. The wall-forming composition was sprayed onto and around the bilayered arrangements in a pan coater until approximately 20 mg of laminate was applied to each tablet.

The trilayered arrangements were coated with a semi-permeable wall. The wall forming composition comprised 99% cellulose acetate having a 39.8% acetyl content and 1% polyethylene glycol comprising a 3.350 viscosity-average molecular weight. The wall-forming composition was dissolved in an acetone:water (95:5 wt:wt) co solvent to make a 5% solids solution. The wall-forming composition was sprayed onto and around the bilayered arrangements in a pan coater until approximately 20 mg of membrane was applied to each tablet.

Next, two 30 mil (0.76 mm) exit passageways were laser drilled through the semi-permeable wall to connect the drug layer with the exterior of the dosage system. The residual solvent was removed by drying for 144 hours as 45 Deg C. and 45% humidity. After drilling, the osmotic systems were dried for 4 hours at 45 Deg C. to remove excess moisture.

Next, the dried systems were overcoated with the drug-containing solution. The solution included risperidone, hydroxypropyl methylcellulose, and citric acid 1.31/97.43/1.26 wt/wt, respectively. The components were dissolved in water resulting in a solution with 7% solids. The drug overcoat composition was sprayed onto and around the dried systems in a pan coater until approximately 8 mg of overcoat was applied to each tablet. The tablets were dried in the coater after drug overcoating.

The dosage form produced by this manufacture was designed to deliver 2 mg of paliperidone in two modes: 0.1 mg as immediate release from the drug overcoat and 1.9 mg in an ascending delivery pattern from two drug-containing cores. The first core contained 1.3% paliperidone, 72.65% polyethylene oxide possessing a 200,000 molecular weight, 20% sodium chloride, USP, 5% hydroxypropylmethyl cellulose having an average viscosity of 5 cps, 0.05% butylated hydroxytoluene, and 1% stearic acid. The second drug core contained 3.1% paliperidone, 90.85% polyethylene oxide possessing a 200,000 molecular weight, 5% hydroxypropylmethyl cellulose having an average viscosity of 5 cps, 0.05% butylated hydroxytoluene, and 1% stearic acid. The push composition comprised 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The semi permeable wall was comprised of 99% cellulose acetate of 39.8% acetyl content and 1% polyethylene glycol. The dosage form comprised two passageways, 30 mils (0.76 mm) on the center of the drug side.

### Example 10

### Risperidone Capsule Shaped Tablet, Trilayer 2 mg System, Slow

A dosage form adapted, designed and shaped as an osmotic drug delivery device was manufactured as follows: 130 g of risperidone, 7265 g of polyethylene oxide with average molecular weight of 200,000 (super fine particle size), and 2000 g of sodium chloride, USP were added to a fluid bed granulator bowl. Next a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 100 g of stearic acid.

Next, a second drug compartment composition was prepared as follows: 310 g of paliperidone and 9085 g of polyethylene oxide with average molecular weight of 200,000 (super fine particle size) were added to a fluid bed granulator bowl. Next, a binder solution was prepared by dissolving 400 g of hydroxypropylmethyl cellulose identified as 2910 having an average viscosity of cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 100 g of stearic acid.

Next, a push composition was prepared as follows: first, a binder solution was prepared. 15.6 kg of polyvinylpyrrolidone identified as K29-32 having an average molecular weight of 40,000 was dissolved in 104.4 kg of water. Then, 24 kg of sodium chloride and 1.2 kg of ferric oxide were sized using a Quadro Comil with a 21-mesh screen. Then, the screened materials and 88.44 kg of Polyethylene oxide (approximately 7,000,000 molecular weight) were added to a fluid bed granulator bowl. The dry materials were fluidized and mixed while 46.2 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 15 g of butylated hydroxytoluene and lubricated with 294 g magnesium stearate.

Next, the paliperidone drug compositions for the first and the second compartments and the push composition were compressed into trilayer tablets. First, 50 mg of the paliperidone compartment one composition was added to the die cavity and pre-compressed, then 40 mg of the paliperidone compartment two composition was added to the die cavity and pre-compressed, then 110 mg of the push composition was added and the layers were pressed into a 3/16″ diameter longitudinal, deep concave, trilayer arrangement.

The trilayered arrangements were coated with a subcoat laminate. The wall forming composition comprised 70% hydroxypropyl cellulose identified as EF, having an average molecular weight of 80,000 and 30% of polyvinylpyrrolidone identified as K29-32 having an average molecular weight of 40,000. The wall-forming composition was dissolved in anhydrous ethyl alcohol, to make an 8% solids solution. The wall-forming composition was sprayed onto and around the bilayered arrangements in a pan coater until approximately 20 mg of laminate was applied to each tablet.

The trilayered arrangements were coated with a semi-permeable wall. The wall forming composition comprised 99% cellulose acetate having a 39.8% acetyl content and 1% polyethylene glycol comprising a 3.350 viscosity-average molecular weight. The wall-forming composition was dissolved in an acetone:water (95:5 wt:wt) co solvent to make a 5% solids solution. The wall-forming composition was sprayed onto and around the bilayered arrangements in a pan coater until approximately 40 mg of membrane was applied to each tablet.

Next, two 30 mil (0.76 mm) exit passageways were laser drilled through the semi-permeable wall to connect the drug layer with the exterior of the dosage system. The residual solvent was removed by drying for 144 hours as 45 Deg C. and 45% humidity. After drilling, the osmotic systems were dried for 4 hours at 45 Deg C. to remove excess moisture.

Next, the dried systems were overcoated with the drug-containing solution. The solution included risperidone, hydroxypropyl methylcellulose, and citric acid 1.31/97.43/1.26 wt/wt, respectively. The components were dissolved in water resulting in a solution with 7% solids. The drug overcoat composition was sprayed onto and around the dried systems in a pan coater until approximately 8 mg of overcoat was applied to each tablet. The tablets were dried in the coater after drug overcoating.

The dosage form produced by this manufacture was designed to deliver 2 mg of paliperidone in two modes: 0.1 mg as immediate release from the drug overcoat and 1.9 mg in an ascending delivery pattern from two drug-containing cores. The first core contained 1.3% paliperidone, 72.65% polyethylene oxide possessing a 200,000 molecular weight, 20% sodium chloride, USP, 5% hydroxypropylmethyl cellulose having an average viscosity of 5 cps, 0.05% butylated hydroxytoluene, and 1% stearic acid. The second drug core contained 3.1% paliperidone, 90.85% polyethylene oxide possessing a 200,000 molecular weight, 5% hydroxypropylmethyl cellulose having an average viscosity of 5 cps, 0.05% butylated hydroxytoluene, and 1% stearic acid. The push composition comprised 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The semi permeable wall was comprised of 99% cellulose acetate of 39.8% acetyl content and 1% polyethylene glycol. The dosage form comprised two passageways, 30 mils (0.76 mm) on the center of the drug side.

### Example 11

### Study to Characterize the Absorption of Risperidone Administered Colonically and Orally in Healthy Volunteers

The study investigated the absorption of risperidone administered colonically and orally in healthy volunteers. The objective of the study was to characterize colonic absorption of risperidone by comparing the AUC_{inf} values of risperidone, paliperidone (a risperidone metabolite) and the active moiety for the colonic treatments and the oral treatment. This was a single-center, two-sequence, open-label, three-treatment, three-period, randomized, crossover pilot study in healthy males. Twelve subjects were dosed with risperidone to ensure that at least 9 subjects completed all three treatments.

Each subject was to receive the following three treatments:
TREATMENT A—2 mg risperidone (50 ml of 0.04 mg/mL solution in water for injection) infused over 6 hours in the transverse colon
TREATMENT B—2 mg risperidone (50 ml of 0.04 mg/mL solution in water for injection) administered as a bolus (administered over ˜10 minutes) in the transverse colon
TREATMENT C—2 mg risperidone (50 ml of 0.04 mg/mL solution in water for injection) administered orally as a bolus

Subjects received the two colonic treatments in the first two periods; the oral treatment was planned to be the last treatment (Period 3). The nasoenteral tube was removed after dosing in each of the two colonic treatments. If in either of the colonic treatments the nasoenteral tube did not reach the colon, the tube was to be removed and the subject was to complete the oral treatment if he had not already received it. A colonic treatment could be attempted again in Period 3 if needed.

If after 6 days, the nasoenteral tube in either of the colonic treatments reached only the ascending colon, drug solution was to be administered into the ascending colon. If the subject received drug solution in the ascending colon during the first colonic treatment, attempts were to be made to administer the drug solution into the ascending colon during the second colonic treatment. The washout period between each treatment was minimum of 6 days and not more than 14 days. The washout period began at the end of dosing. Twenty blood samples were collected from each subject for measurement of risperidone plasma concentrations during each treatment session. Samples were obtained at 0 (pre-dose), 0.25, 0.5, 0.75, 1, 1.5, 2, 3, 4, 5, 6, 7, 8, 12, 24, 30, 36, 48, 54, and 60 hours after dosing.

Pharmacokinetic parameters such as AUCt, AUCinf, Cmax, Tmax, and t1/2 were calculated for risperidone and paliperidone and for the active moieties (i.e. sum of the two analytes risperidone+paliperidone) for each treatment and subject. Relative Bioavailability was estimated for the colonic treatments. A summary of the observed values of these parameters is provided in Table 3.

The bioavailability of risperidone following the 6-hour colonic infusion and the 10-minute colonic bolus relative to oral dosing was 75% and 63%, respectively. Relative bioavailability compared to oral dosing was estimated as follows:

The bioavailability of paliperidone following the 6-hour colonic infusion and the 10-minute colonic bolus relative to oral dosing was 55% and 51%, respectively. The bioavailability of active moiety (sum of risperidone and its metabolite, paliperidone) following the 6-hour risperidone colonic infusion and the 10-minute colonic bolus relative to oral dosing was 60% and 53%, respectively.

Mean drug-to-metabolite ratio of the AUC_{inf} values were similar in all three treatments, suggesting drug metabolism is similar following oral and colonic delivery (0.26, 0.33, and 0.31, for the oral solution, colonic infusion over 6 hours, and colonic bolus over 10 minutes, respectively).

TABLE 3
Mean Pharmacokinetic Parameter Values

| | Treatment A | | Treatment B | | Treatment C | |
|---|---|---|---|---|---|---|
| | Colonic infusion | | Colonic bolus | | 2 mg Oral | |
| | 2 mg over 6 hours | | 2 mg over 10 minutes | | solution | |
| | n | | | | | |
| Parameters | 11 | | 11 | | 12 | |
| | Mean (SD) Risperidone Pharmacokinetics | | | | | |
| Cₘₐₓ(ng/mL) | 4.93 | (4.26) | 7.31 | (5.04) | 16.24 | (7.60) |
| Tₘₐₓ(h) | 6.18 | (0.87) | 0.77 | (0.21) | 0.69 | (0.16) |
| t_{1/2}(h) | 4.11 | (4.01)³ | 3.32 | (2.83) | 3.31 | (1.56) |
| AUCₜ(ng · h/mL) | 53.1 | (73.2) | 29.7 | (19.8) | 58.7 | (29.8) |
| AUC_{inf}(ng · h/mL) | 56.1 | (78.0) | 30.6 | (20.1) | 59.7 | (30.0) |
| Relative Bioavailability | 75.0% | (74.0) | 63.4% | (44.8) | NA (reference | |
| | | | | | treatment) | |
| | Mean (SD) Paliperidone Pharmacokinetics | | | | | |
| Cₘₐₓ(ng/mL) | 4.53 | (2.96) | 5.33 | (3.77) | 9.49 | (5.09) |
| Tₘₐₓ(h) | 10.83 | (8.56) | 3.91 | (1.76) | 5.50 | (2.68) |
| t_{1/2}(h) | 19.8 | (8.6)³ | 16.9 | (4.6) | 22.4 | (9.9) |
| AUCₜ(ng · h/mL) | 113.7 | (67.9) | 109.7 | (70.2) | 211.7 | (79.6) |
| AUC_{inf}(ng · h/mL) | 134.7 | (83.7) | 119.0 | (73.8) | 243.5 | (89.0) |
| Relative Bioavailability | 54.6% | (29.0) | 50.8% | (28.0) | NA(reference | |
| | | | | | treatment) | |
| | Mean (SD) Active Moiety | | | | | |
| Cₘₐₓ(ng/mL)¹ | 9.10 | (5.93) | 11.24 | (7.02) | 23.42 | (9.66) |
| Tₘₐₓ(h)¹ | 7.01 | (0.04) | 0.96 | (0.33) | 0.78 | (0.20) |
| AUCₜ(ng · h/mL)² | 166.8 | (117.4) | 139.4 | (84.3) | 270.4 | (93.2) |
| AUC_{inf}(ng · h/mL)² | 190.8 | (144.0) | 149.6 | (88.0) | 303.1 | (104.2) |
| Relative Bioavailability | 60.0% | (40.4) | 52.5% | (29.5) | NA(reference | |
| | | | | | treatment) | |
| | Mean (SD) Drug AUC_{inf}/Metabolite | | | | | |
| | AUC_{inf} Ratio | | | | | |
| Risperidone | 0.33 | (0.33) | 0.31 | (0.26) | 0.26 | (0.13) |
| AUC_{inf}/ | | | | | | |
| 9-hydroxyrisperidone AUC_{inf} | | | | | | |

¹Cₘₐₓ and Tₘₐₓ values estimated from the concentration profile of the sum of risperidone and paliperidone
²AUC values estimated by sum of AUC values of risperidone and paliperidone
³n = 10 for risperidone and n = 9 for paliperidone

### Example 12

### Pharmacokinetics of Paliperidone and Risperidone when Administered as Osmotic Modules and Oral Solutions in Healthy Volunteers

The study investigated the pharmacokinetics of single doses of paliperidone and risperidone following administration of oral solution and in a prototype controlled release formulation (osmotic modules). This was a single-center, open-label, randomized, four-treatment, four-sequence, four-period, crossover pilot study in healthy males and females to characterize the pharmacokinetics of paliperidone and risperidone when administered as osmotic modules and oral solutions. Sixteen subjects were to be dosed with paliperidone and risperidone to ensure that at least 12 subjects completed the study.

Each subject received 2 mg risperidone, and 2 mg paliperidone according to the following four treatments:
TREATMENT A—Osmotic module (2 mg risperidone, prepared according to Example 1)
TREATMENT B—Solution (2 mg risperidone) administered orally as a bolus
TREATMENT C—Osmotic module (2 mg paliperidone, prepared according to Example 2)
TREATMENT D—Solution (2 mg paliperidone) administered orally as a bolus

Subjects received both risperidone treatments before receiving the paliperidone treatments. Treatments were separated by a washout period of not less than 6 days and not more than 14 days. The washout period began 24 h after dosing. Sixteen subjects were enrolled in the study, and one subject withdrew 8 days after the second study period. During the osmotic module treatments, fifteen blood samples (7 mL each sample) were collected from each subject for measurement of risperidone and paliperidone (risperidone treatment), or paliperidone (paliperidone treatment) plasma concentrations. Samples were obtained at 0 (pre-dose), 1, 2, 4, 6, 9, 12, 15, 16, 18, 24, 36, 48, 72, and 96 hours post dose.

During the oral solution treatments, fifteen blood samples (7 mL each sample) were collected from each subject for measurement of risperidone and paliperidone (risperidone treatment), or paliperidone (paliperidone treatment) plasma concentrations. Samples were obtained at 0 (pre-dose), 0.5, 1, 1.5, 2.5, 4, 6, 9, 12, 18, 24, 36, 48, 72, and 96 hours post dose.

PK parameters AUCₜ, AUC_{inf}, Cₘₐₓ, Tₘₐₓ, and t_{1/2} were calculated for paliperidone for each treatment and subject. Risperidone and active moiety (risperidone+paliperidone) parameters were estimated for the two risperidone treatments.

Sixteen subjects completed risperidone treatments (Treatments A and B), and 15 subjects completed paliperidone treatments (Treatments C and D). Tables 4 and 5 present the summary of the mean pharmacokinetic parameters.

The osmotic module treatment resulted in a much lower Cₘₐₓ and provided later peaks (Tₘₐₓ) compared to the oral solution treatment of each drug.

The relative bioavailability (BA) of risperidone, paliperidone, and active moiety following risperidone osmotic module dosing relative to oral solution was 59.6%, 67.1%, and 65.6%, respectively. The BA of paliperidone osmotic module relative to the oral solution was 62.5%.

The drug-to-metabolite ratios were similar following administration of risperidone via osmotic module and oral solution, which suggests that the drug metabolism is similar between the two formulations.

The AUC and relative BA of the active moiety following risperidone are similar to the AUC and relative BA following paliperidone for both formulations.

TABLE 4
Pharmacokinetic Data following Risperidone Treatments (A and B; n = 16)

| | 2 mg | | 2 mg | | 2 mg | | 2 mg | | 2 mg | | 2 mg | |
|---|---|---|---|---|---|---|---|---|---|---|---|---|
| | Risperidone | | Risperidone | | Risperidone | | Risperidone | | Risperidone | | Risperidone | |
| | Osmotic Module | | Oral | | Osmotic Module | | Oral | | Osmotic Module | | Oral | |
| Parameters | Risperidone | | Risperidone | | Paliperidone | | Paliperidone | | Active Moiety | | Active Moiety | |
| Cₘₐₓ(ng/mL) | 4.28 | (2.50) | 15.57 | (6.23) | 4.35 | (1.36) | 8.48 | (3.64) | 7.83 | (1.80)¹ | 21.80 | (5.46)¹ |
| Tₘₐₓ(h) | 6.63 | (2.34) | 0.99 | (0.23) | 15.20 | (6.02) | 6.63 | (5.44) | 8.13 | (2.50)¹ | 1.87 | (2.06)¹ |
| t_{1/2}(h) | 7.6 | (5.9)³ | 6.5 | (6.4)³ | 24.4 | (6.3) | 27.1 | (9.4)³ | — | | — | |
| AUCₜ(ng · h/mL) | 77.3 | (81.3) | 128.2 | (127.9) | 166.5 | (46.0) | 260.9 | (83.9) | 243.7 | (79.7)² | 389.1 | (142.6)² |
| AUC_{inf}(ng · h/mL) | 79.6 | (83.4) | 136.1 | (145.2) | 182.4 | (47.7) | 283.7 | (83.8) | 262.0 | (87.6)² | 419.8 | (169.0)² |

| | | | | | | | | | |
|---|---|---|---|---|---|---|---|---|---|
| Relative Bioavailability Module/Oral | 59.6% | (18.8%) | N/A | 67.1% | (15.6%) | N/A | 65.6% | (16.7%) | N/A |

Dose Normalized Parameters

| | | | | | | | | | | | | |
|---|---|---|---|---|---|---|---|---|---|---|---|---|
| Cₘₐₓ(ng/mL/mg) | 2.14 | (1.25) | 7.8 | (3.15) | 2.2 | (0.68) | 4.24 | (1.82) | 3.92 | (0.9) | 10.9 | (2.73) |
| AUC_{inf}(ng · h/mL/mg) | 39.8 | (41.7) | 68.1 | (72.6) | 91.2 | (23.9) | 141.9 | (41.9) | 131.0 | (43.8) | 209.9 | (84.5) |

¹Cₘₐₓ and Tₘₐₓ values estimated from concentration profile of the sum of risperidone and paliperidone
²AUC values estimated by sum of AUC values of risperidone and paliperidone
³n = 15

TABLE 5
Pharmacokinetic Data following Paliperidone
Treatments (C and D; n = 15)

| | 2 mg Paliperidone | | 2 mg Paliperidone | |
|---|---|---|---|---|
| Parameters | Osmotic module | | Oral solution | |
| Cₘₐₓ(ng/mL) | 6.39 | (1.91) | 17.72 | (4.47) |
| Tₘₐₓ(h) | 11.27 | (3.4) | 1.31 | (0.59) |
| t_{1/2}(h) | 27.5 | (4.3) | 29.3 | (9.9)¹ |
| AUCₜ(ng · h/mL) | 246.0 | (75.4) | 399.6 | (103.8) |
| AUC_{inf}(ng · h/mL) | 271.4 | (81.8) | 439.4 | (128.6) |

| | | | |
|---|---|---|---|
| Relative Bioavailability | 62.5% | (11.3%) | N/A |
| Module/oral | | | |

Dose Normalized Parameters

| | | | | |
|---|---|---|---|---|
| Cₘₐₓ(ng/mL) | 3.2 | (0.96) | 8.9 | (2.24) |
| AUC_{inf}(ng · h/mL) | 135.7 | (40.9) | 219.7 | (64.3) |

¹n=13

### Example 13

### Evaluation of OROS® (paliperidone) Pharmacokinetics and Pharmacodynamics

This study investigated the pharmacokinetics and pharmacodynamic effects (postural changes in blood pressure and heart rate) of 2 different formulations of OROS® (paliperidone) and compared with oral paliperidone solution and also evaluated the effect of food on the pharmacokinetics of SLOW OROS® (paliperidone).

This was a single-center, single-dose, open-label, randomized, 4-treatment, 4-sequence, 4-period, crossover study. Each subject received the following 4 treatments in a random manner (all doses refer to the total drug content in the formulation):
TREATMENT A—FAST OROS® (paliperidone), 4 mg (2×2 mg, prepared according to Example 8), Fasted
TREATMENT B—SLOW OROS® (paliperidone), 4 mg (2×2 mg, prepared according to Example 7), Fasted
TREATMENT C—SLOW OROS® (paliperidone), 4 mg (2×2 mg, prepared according to Example 7), with Food (FDA-standard high-fat breakfast; about half of the breakfast's ˜1000 calories were provided by fat)
TREATMENT D—Immediate-release (IR) Oral Solution paliperidone (in Tartaric acid solution at approximately pH 6.9-7.1), 2 mg, Fasted

Twenty-seven subjects received all 4 study treatments. The FAST OROS® (paliperidone) system was designed to release the dose over approximately 14 hours; the SLOW OROS® (paliperidone) system was designed to release the dose over approximately 24 hours. There was a 6- to 14-day washout period between treatments, which began 24 hours after dosing in each treatment period. During each treatment, blood samples were collected from each subject to determine plasma paliperidone concentrations. Samples were collected at:

FAST OROS® (paliperidone): 0 (pre-dose), 2, 4, 6, 8, 10, 11, 12, 13.5, 16, 18, 22, 24, 27, 30, 36, 42, 48, 58, 72, and 96 hours post dose for

SLOW OROS® (paliperidone): 0 (pre-dose), 2, 4, 6, 9, 12, 16, 18, 20, 22, 24, 27, 30, 33, 36, 42, 48, 58, 72, and 96 hours post dose

IR Oral Solution paliperidone treatment: 0 (pre-dose), 0.5, 1, 1.5, 2, 3, 4, 6, 9, 12, 18, 24, 36, 48, 58, 72, and 96 hours post dose.

Postural changes in blood pressure and heart rate were assessed with an automated blood pressure monitor during each treatment at 0 (pre-dose), 1, 2, 4, 8, 10, 12, 16, 20, 22, 24, 36, 48, 72, and 96 hours post dose. Two supine blood pressure and heart rate measurements were collected. At 2 and 3 minutes after standing from the supine position, blood pressure and heart rate were again measured. Dizziness and fainting symptoms after standing were assessed.

PK parameters AUCₜ, AUC_{inf}, Cₘₐₓ, Tₘₐₓ, and t_{1/2} were calculated for paliperidone for each treatment and subject.

The percentage of subjects with >20 mm Hg drop in systolic blood pressure (SBP) at 3 minutes of standing or with symptoms of orthostatic hypotension (dizziness or faintness) was summarized for Days 1 and 2

Pharmacokinetic parameters as well as ratios and 90% Cls are summarized in Table 6. The SLOW OROS® treatments (fasted and fed) resulted in a lower Cₘₐₓ and provided later peaks (Tₘₐₓ) compared with IR Oral Solution paliperidone. FAST OROS® treatment also resulted in a lower Cₘₐₓ and provide later peaks (Tₘₐₓ) compared with the IR Oral Solution paliperidone, but to a lesser degree than the SLOW OROS® treatments (fasted and fed).

Mean bioavailability estimated for FAST OROS® (paliperidone) and SLOW OROS® (paliperidone) in the fasted state was 52% and 34%, respectively, relative to IR Oral Solution. Mean bioavailability of SLOW OROS® in the fed state (40%) was higher than in the fasted state.

TABLE 6
Paliperidone Pharmacokinetic Parameters, Mean (CV), n = 27

| | FAST | | SLOW | | SLOW | | IR Oral | |
|---|---|---|---|---|---|---|---|---|
| | OROS ® Fasted | | OROS ® Fasted | | OROS ® Fed | | Solution | |
| Parameter | 4 mg | | 4 mg | | 4 mg | | Fasted 2 mg | |
| Cₘₐₓ (ng/mL) | 12.2 | (35%) | 6.7 | (53%) | 8.2 | (61%) | 19.4 | (34%) |
| Tₘₐₓ (h) | 11.4 | (18%) | 22.2 | (17%) | 22.7 | (16%) | 1.2 | (47%) |
| t_{1/2} (h) ^{a} | 25.95 | (15%) | 28.17 | (29%) | 25.81 | (21%) | 26.98 | (19%) |
| AUC₍₀₋₉₆₎ | 372 | (37%) | 243 | (50%) | 285 | (54%) | 371 | (36%) |
| (ng · h/mL) | | | | | | | | |
| AUC_{inf} | 403 | (37%) | 272 | (50%) | 314 | (54%) | 397 | (36%) |
| (ng · h/mL) | | | | | | | | |

| | | | | | | | |
|---|---|---|---|---|---|---|---|
| Bioavailability (%) | 52 | (31%) | 34 | (31%) | 40 | (48%) | Reference |

| Range | 1-74 | 9-61 | 23-91 | | |
|---|---|---|---|---|---|
| AUC_{inf} | 45% | 32% | 36% | | Reference |
| Ratio (90% Cl) ^{b} | (36%-56%) | (26%-40%) | (29%-45%) | | |
| Cₘₐₓ Ratio | NA | Reference | 115% | (93%-143%) | NA |
| (90% Cl) ^{b} | | | | | |
| AUC_{inf} Ratio | NA | Reference | 111% | (89%-139%) | NA |
| (90% Cl) ^{b} | | | | | |

Dose Normalized Cₘₐₓ and AUC

| | | | | |
|---|---|---|---|---|
| Cₘₐₓ | 3.05 | 1.7 | 2.05 | 9.7 |
| (ng/mL/mg) | | | | |
| AUC_{inf} | 101 | 68 | 78.5 | 199 |
| (ng · h/mL/mg) | | | | |

^{a} n = 25 for FAST OROS ® Fasted; n = 26 for SLOW OROS ® Fasted
^{b} Based on log-transformed analysis
NA = not applicable

### Example 14

### Evaluation of OROS®(Risperidone) and IR Risperidone Pharmacokinetics

This study investigated the pharmacokinetics of 2 different formulations of OROS®(risperidone) and compared with IR risperidone and also evaluated the effect of food on the pharmacokinetics of SLOW OROS®(risperidone).

This was a single-center, single-dose, open-label, randomized, 4-treatment, 4-sequence, 4-period, crossover study. Each subject received the following 4 treatments in a random manner (all doses refer to the total drug content in the formulation):
TREATMENT A: FAST OROS® (Risperidone), 2 mg, prepared as in Example 9, fasted.
TREATMENT B: SLOW OROS® (Risperidone), 2 mg, prepared as in Example 10, fasted.
TREATMENT C: SLOW OROS® (Risperidone), 2 mg, prepared as in Example 10, with food.
TREATMENT D: Immediate Release Risperidone, 2 mg (IR-2) fasted.

Thirty-two healthy males and females were enrolled and 24 subjects received all four study treatments. FAST OROS® and SLOW OROS® were designed to deliver the doses in approximately 14 hours and 24 hours, respectively. There was a 6- to 14-day washout period between treatments, which began 24 hours after dosing in each treatment period. During each treatment, blood samples were collected from each subject to determine plasma paliperidone concentrations. Samples were collected at:

FAST OROS® (Risperidone) 2 mg fasted: 0 (pre-dose), 1, 2, 4, 6, 8, 10, 11, 12, 13.5, 15, 18, 21, 24, 27, 30, 36, 42, 48, 58, 72, and 96 hours (h) after treatment initiation.

SLOW OROS® (Risperidone) 2 mg fasted the blood draw times were: 0 (pre-dose), 1, 2, 4, 6, 9, 12, 16, 18, 20, 22, 24, 27, 30, 33, 36, 42, 48, 58, 72, and 96 h after treatment initiation.

IR-2 dosing, the blood draw times were: 0 (pre-dose), 0.5, 1, 1.5, 3, 4, 6, 9, 12, 18, 24, 36, 48, 58, 72, and 96 hours h after treatment initiation.

PK parameters AUCₜ, AUC_{inf}, Cₘₐₓ, Tₘₐₓ, and t_{1/2} were calculated for paliperidone for each treatment and subject.

Pharmacokinetic parameters as well as ratios and 90% Cls are summarized in Table 7.

The SLOW OROS® treatments (fasted and fed) resulted in a lower Cₘₐₓ and provided later peaks (Tₘₐₓ) compared with IR risperidone. FAST OROS® treatment also resulted in a lower Cₘₐₓ and provide later peaks (Tₘₐₓ) compared with the IR risperidone, but to a lesser degree than the SLOW OROS® treatments (fasted and fed). Mean half-life for risperidone and paliperidone values were similar among the four treatments.

Mean bioavailability estimated for FAST OROS® (risperidone) and SLOW OROS® (risperidone) in the fasted state for the three analytes was in the range of 52 to 55% and 41 to 42%, respectively, relative to IR-2 mg risperidone. Mean bioavailability of SLOW OROS® in the fed state (48 to 49%) was higher than in the fasted state (41 to 42%). The results of the ANOVA and 90% confidence intervals are also presented in Table 7.

TABLE 7
Pharmacokinetic Values Following Four Risperidone Treatments

| | | | | |
|---|---|---|---|---|
| | FAST OROS ® | SLOW OROS ® | SLOW OROS ® | Risperidone |
| | (Risperidone) | (Risperidone) | (Risperidone) | IR-2 mg |
| N = 24 | 2 mg (fasted) | 2 mg (fasted) | 2 mg (with food) | (fasted) |

Risperidone Concentration Mean (SD)

| | | | | | | | | |
|---|---|---|---|---|---|---|---|---|
| Cₘₐₓ (ng/mL) | 3.2 | (2.5) | 1.7 | (1.6) | 2.1 | (2.2) | 15.3 | (7.0) |
| Tₘₐₓ (h) | 9.1 | (2.8) | 15.2 | (10.7) | 14.6 | (8.1) | 1.1 | (0.5) |
| T_{1/2} (h) | 7.6 | (7.9) | 9.1 | (10.8)^{b} | 8.6 | (6.2)^{c} | 9.1 | (16.0) |
| AUCt (ng · h/mL) | 57.4 | (74.1) | 44.7 | (55.3) | 52.4 | (67.9) | 107.2 | (121.2) |
| AUC (0-96) (ng · h/mL) | 57.9 | (74.1) | 44.9 | (55.2) | 53.1 | (68.6) | 107.9 | (121.1) |
| AUCinf (ng · h/mL) | 60.2 | (78.6) | 47.4 | (59.7) | 54.9 | (70.2) | 113.4 | (134.4) |

| | | | | | | | |
|---|---|---|---|---|---|---|---|
| Bioavailability | 52.4 | (15.0) | 41.1 | (19.1) | 48.5 | (21.3) | Reference |

Dose Normalized Cmax and AUC

| | | | | | | | | |
|---|---|---|---|---|---|---|---|---|
| Cₘₐₓ (ng/mL/mg) | 1.6 | (1.25) | 0.85 | (0.8) | 1.05 | (1.1) | 7.7 | (3.5) |
| AUC_{inf} | 30.1 | (39.3) | 23.7 | (29.9) | 27.5 | (35.1) | 56.7 | (67.2) |
| (ng · h/mL/mg) | | | | | | | | |
| ANOVA | | | | | | | | |

| | | | | | | | |
|---|---|---|---|---|---|---|---|
| AUC_{inf} (vs IR-2) Ratio | 50.2% | (43.9, 57.3) | 37.9% | (33.2, 43.4) | 45.4% | (39.6, 52.0) | Reference |
| (90% Cl)^{a} | | | | | | | |

| | | | | | |
|---|---|---|---|---|---|
| Cₘₐₓ (vs SLOW fasted) | NA | Reference | 119.1% | (99.2, 143.0) | NA |
| Ratio^{a} (90% Cl) | | | | | |
| AUC_{inf} vs SLOW fasted) | NA | Reference | 119.6% | (104.5, 136.9) | NA |
| Ratio^{a} (90% Cl) | | | | | |

Paliperidone Concentration Mean (SD)

| | | | | | | | | |
|---|---|---|---|---|---|---|---|---|
| Cₘₐₓ (ng/mL) | 3.8 | (2.8) | 2.3 | (1.4) | 2.7 | (1.6) | 8.6 | (4.8) |
| Tₘₐₓ (h) | 15.1 | (6.1) | 26.4 | (4.3) | 24.6 | (5.8) | 5.6 | (5.9) |
| T_{1/2} (h) | 27.4 | (5.3)^{d} | 26.1 | (6.2) | 25.7 | (6.4) | 28.2 | (6.8)^{b} |
| AUCt (ng · h/mL) | 118.0 | (65.8) | 88.8 | (44.1) | 100.7 | (56.4) | 212.7 | (95.9) |
| AUC(0-96) (ng · h/mL) | 118.3 | (65.8) | 88.9 | (44.1) | 101.3 | (55.8) | 212.7 | (95.9) |
| AUCinf (ng · h/mL) | 130.3 | (68.3) | 98.7 | (46.7) | 112.8 | (62.2) | 232.8 | (99.5) |

| | | | | | | | |
|---|---|---|---|---|---|---|---|
| Bioavailability | 55.1 | (12.7) | 42.4 | (10.6) | 48.0 | (19.2) | Reference |

Dose Normalized Cmax and AUC

| | | | | | | | | |
|---|---|---|---|---|---|---|---|---|
| Cₘₐₓ | 1.9 | (1.4) | 1.2 | (0.7) | 1.35 | (0.8) | 4.3 | (2.4) |
| (ng/mL/mg) | | | | | | | | |
| AUC_{inf}(ng · h/mL/mg) | 65.2 | (34.2) | 49.4 | (23.4) | 56.4 | (31.1) | 116.4 | (49.8) |
| ANOVA | | | | | | | | |

| | | | | | | | |
|---|---|---|---|---|---|---|---|
| AUC_{inf} (vs IR-2) Ratio | 54.3% | (48.8, 60.4) | 41.2% | (37.1, 45.9) | 45.8% | (41.2, 51.1) | Reference |
| (90% Cl)^{a} | | | | | | | |

| | | | | | |
|---|---|---|---|---|---|
| Cₘₐₓ (vs SLOW fasted) | NA | Reference | 119.5% | (100.5, 142.0) | NA |
| Ratio (90% Cl)^{a} | | | | | |
| AUC_{inf} (vs SLOW fasted) | NA | Reference | 111.2% | (99.9, 123.9) | NA |
| Ratio^{a} (90% Cl) | | | | | |

Active Moiety Concentration^{e} Mean (SD)

| | | | | | | | | |
|---|---|---|---|---|---|---|---|---|
| Cₘₐₓ (ng/mL) | 6.6 | (3.1) | 3.8 | (1.8) | 4.6 | (2.2) | 22.0 | (6.2) |
| Tₘₐₓ (h) | 11.0 | (2.1) | 22.1 | (5.7) | 20.9 | (6.3) | 1.1 | (0.5) |
| AUCt (ng · h/mL) | 175.4 | (82.8) | 133.5 | (59.9) | 153.1 | (76.0) | 319.9 | (109.3) |
| AUC(0-96) (ng · h/mL) | 176.2 | (82.6) | 133.8 | (59.8) | 154.4 | (76.5) | 320.6 | (109.1) |
| AUCinf (ng · h/mL) | 190.5 | (88.3) | 146.1 | (66.9) | 167.7 | (82.7) | 346.2 | (126.1) |

| | | | | | | | |
|---|---|---|---|---|---|---|---|
| Bioavailability | 54.5 | (12.8) | 41.8 | (11.1) | 48.4 | (19.4) | Reference |

Dose Normalized Cmax and AUC

| | | | | | | | | |
|---|---|---|---|---|---|---|---|---|
| Cₘₐₓ | 3.3 | (1.6) | 1.9 | (0.9) | 2.3 | (1.1) | 11 | (3.1) |
| (ng/mL/mg) | | | | | | | | |
| AUC_{inf} (ng · h/mL/mg) | 95.3 | (44.2) | 73.1 | (33.5) | 83.9 | (41.4) | 173.1 | (63.1) |
| ANOVA | | | | | | | | |

| | | | | | | | |
|---|---|---|---|---|---|---|---|
| AUC_{inf} (vs IR-2) Ratio | 53.4% | (47.9, 59.5) | 40.7% | (36.5, 45.3) | 46.3% | (41.5, 51.7) | Reference |
| (90% Cl)^{a} | | | | | | | |

| | | | | | |
|---|---|---|---|---|---|
| Cₘₐₓ (vs SLOW fasted) | NA | Reference | 121.4% | (104.0, 141.7) | NA |
| Ratio^{a} (90% Cl) | | | | | |
| AUC_{inf} vs SLOW fasted) | NA | Reference | 114.0% | (102.1, 127.2) | NA |
| Ratio^{a} (90% Cl) | | | | | |

^{a}Used a log transformation and ANOVA
^{b}n = 22;
^{c}n = 21;
^{d}n = 23
^{e}Cmax and Tmax values estimated from the concentration profile of the sum of risperidone and paliperidone.
AUC values estimated by sum of AUC values of risperidone and paliperidone.

### Example 15

### 2 mg Risperidone Formulation with Enteric Coating

First, a liquid formulation is prepared as follows: 29.862 g of polyoxyethylene-polyoxypropylene copolymer (Poloxamer L-44) is weighed into the glass jar. Then, 15 mg of butylated hydroxytoluene is mixed with polysorbate 80 for 30 seconds. Finally, 0.123 g of risperidone is added into solution, pre-mixed with a spatula for 30 seconds and then mixed on a stirring plate for 20 hours.

Next, an empty HPMC capsule is filled with the liquid formulation using a syringe. Approximately 500 mg of the liquid formulation is dispensed into each capsule. The opening in the capsule created by the syringe is then covered by coating with a 5% solution of cellulose acetate 398-10 in acetone.

Next, the HPMC capsule is coated with an enteric coating. The enteric coating composition comprises 97% of hydroxypropylmethylcellulose phthalate 55S and 3% of triethylcitrate. The enteric coating composition is dissolved in 50/50 acetone/methanol mixture. The enteric coating composition is sprayed onto and around the capsules in a pan coater until approximately 40 mg of enteric coat is applied to each capsule.

The residual solvent is removed by drying for 144 hours as 45 Deg C. and 45% humidity.

What is claimed is:

1. A dosage form comprising:
a controlled release dosing structure; and
a liquid formulation contained within the controlled release dosing structure;
wherein the liquid formulation comprises a benzisoxazole derivative and a liquid carrier.

2. The dosage form of claim 1, wherein the dosage form comprises an osmotic dosage form.

3. The dosage form of claim 1, wherein the benzisoxazole derivative comprises paliperidone or pharmaceutically acceptable salts thereof.

4. The dosage form of claim 1, wherein the benzisoxazole derivative comprises risperidone or pharmaceutically acceptable salts thereof.

5. The dosage form of claim 1, wherein the benzisoxazole derivative is present in an amount ranging from about 0.1 mg to about 20 mg.

6. The dosage form of claim 5, wherein the benzisoxazole derivative is present in an amount ranging from about 0.1 mg to about 5 mg.

7. The dosage form of claim 1, wherein the liquid carrier comprises a lipophilic solvents, a surfactant, or a hydrophilic solvent.

8. The dosage form of claim 1, wherein the liquid formulation further comprises an antioxidant.

9. A method comprising:
providing a dosage form that comprises a controlled release dosing structure;
providing a liquid formulation within the controlled release dosing structure, wherein the liquid formulation comprises a benzisoxazole derivative and a liquid carrier; and
causing the controlled release dosing structure to controllably release the liquid formulation.

10. The method of claim 9, wherein causing the controlled release dosing structure to controllably release the liquid formulation comprises administering the dosage form to a patient.

11. The method of claim 9, wherein the dosage form comprises an osmotic dosage form.

12. The method of claim 9, wherein the benzisoxazole derivative comprises paliperidone or pharmaceutically acceptable salts thereof.

13. The method of claim 9, wherein the benzisoxazole derivative comprises risperidone or pharmaceutically acceptable salts thereof.

14. The method of claim 9, wherein the benzisoxazole derivative is present in an amount ranging from about 0.1 mg to about 20 mg.

15. The method of claim 14, wherein the benzisoxazole derivative is present in an amount ranging from about 0.1 mg to about 5 mg.

16. The method of claim 9, wherein the liquid carrier comprises a lipophilic solvents, a surfactant, or a hydrophilic solvent.

17. The method of claim 9, wherein the liquid formulation further comprises an antioxidant.
